(19) United States
(12) Patent Application Publication
(10) Pub. No.: US 2006/0281778 A1
(43) Pub. Date: Dec. 14, 2006

(54) Compounds for inhibiting KSP Kinesin activity

(75) Inventors: Jayaram R. Tagat, Westfield, NJ (US); Timothy J. Guzi, Chatham, NJ (US); Marc Labroli, Mount Laurel, NJ (US); Cory Poker, Springfield, NJ (US); Angela D. Kerekes, Scotch Plains, NJ (US); Tao Yu, Edison, NJ (US); Hon-Chung Tsui, East Brunswick, NJ (US); Neng-Yang Shih, Warren, NJ (US); Yushi Xiao, Edison, NJ (US); Sunil Paliwal, Monroe Township, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/369,625

(22) Filed: Mar. 7, 2006

Related U.S. Application Data
(60) Provisional application No. 60/660,134, filed on Mar. 9, 2005.

(51) Int. Cl. A61K 31/4745; C07D 498/02

(52) U.S. Cl. 514/292; 546/83

(57) ABSTRACT

The present invention provides compounds of Formula I (wherein R¹, R³, X, W, Z and ring Y are as defined herein). The present invention also provides compositions comprising these compounds that are useful for treating cellular proliferative diseases or disorders associated with KSP kinesin activity and for inhibiting KSP kinesin activity.

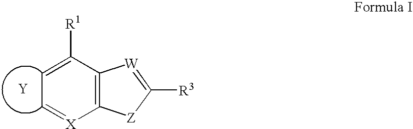

This Application claims the benefit of U.S. Provisional Application Ser. No. 60/660,134, filed Mar. 9, 2005, which is incorporated by reference herein in its entirely.

### FIELD OF THE INVENTION

The present invention relates to compounds and compositions that are useful for treating cellular proliferative diseases or disorders associated with Kinesin Spindle Protein (“KSP”) kinesin activity and for inhibiting KSP kinesin activity.

### BACKGROUND OF THE INVENTION

Cancer is a leading cause of death in the United States and throughout the world. Cancer cells are often characterized by constitutive proliferative signals, defects in cell cycle checkpoints, as well as defects in apoptotic pathways. There is a great need for the development of new chemotherapeutic drugs that can block cell proliferation and enhance apoptosis of tumor cells.

Conventional therapeutic agents used to treat cancer include taxanes and vinca alkaloids, which target microtubules. Microtubules are an integral structural element of the mitotic spindle, which is responsible for the distribution of the duplicated sister chromatids to each of the daughter cells that result from cell division. Disruption of microtubules or interference with microtubule dynamics can inhibit cell division and induce apoptosis.

However, microtubules are also important structural elements in non-proliferative cells. For example, they are required for organelle and vesicle transport within the cell or along axons. Since microtubule-targeted drugs do not discriminate between these different structures, they can have undesirable side effects that limit usefulness and dosage. There is a need for chemotherapeutic agents with improved specificity to avoid side effects and improve efficacy.

Microtubules rely on two classes of motor proteins, the kinesins and dyneins, for their function. Kinesins are motor proteins that generate motion along microtubules. They are characterized by a conserved motor domain, which is approximately 320 amino acids in length. The motor domain binds and hydrolyses ATP as an energy source to drive directional movement of cellular cargo along microtubules and also contains the microtubule binding interface (Mandelkow and Mandelkow, Trends Cell Biol. 2002,12:585-591).

Kinesins exhibit a high degree of functional diversity, and several kinesins are specifically required during mitosis and cell division. Different mitotic kinesins are involved in all aspects of mitosis, including the formation of a bipolar spindle, spindle dynamics, and chromosome movement. Thus, interference with the function of mitotic kinesins can disrupt normal mitosis and block cell division. Specifically, the mitotic kinesin KSP (also termed EG5), which is required for centrosome separation, was shown to have an essential function during mitosis. Cells in which KSP function is inhibited arrest in mitosis with unseparated centrosomes (Blangy et al., Cell 1995, 83:1159-1169). This leads to the formation of a monoastral array of microtubules, at the end of which the duplicated chromatids are attached in a rosette-like configuration. Further, this mitotic arrest leads to growth inhibition of tumor cells (Kaiser et al., J. Biol. Chem. 1999, 274:18925-18931). Inhibitors of KSP would be desirable for the treatment of proliferative diseases, such as cancer.

Kinesin inhibitors are known, and several molecules have recently been described in the literature. For example, adociasulfate-2 inhibits the microtubule-stimulated ATPase activity of several kinesins, including CENP-E (Sakowicz et al., Science 1998, 280:292-295). Rose Bengal lactone, another non-selective inhibitor, interferes with kinesin function by blocking the microtubule binding site (Hopkins et al., Biochemistry 2000, 39:2805-2814). Monastrol, a compound that has been isolated using a phenotypic screen, is a selective inhibitor of the KSP motor domain (Mayer et al., Science 1999, 286:971-974). Treatment of cells with monastrol arrests cells in mitosis with monopolar spindles.

KSP, as well as other mitotic kinesins, are attractive targets for the discovery of novel chemotherapeutics with anti-proliferative activity. There is a need for compounds useful in the inhibition of KSP, and in the treatment of proliferative diseases, such as cancer.

### SUMMARY OF THE INVENTION

In one embodiment, the present invention provides a compound represented by the structural Formula I: 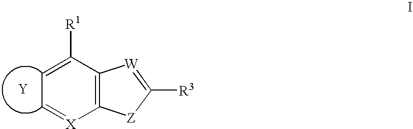
or a pharmaceutically acceptable salt, solvate or ester thereof, wherein:

ring Y is a 5- to 6-membered aryl or a 5- or 6-membered heteroaryl fused as shown in Formula I, wherein in said aryl and heteroaryl each substitutable ring carbon is independently substituted with R² and each substitutable ring nitrogen is independently substituted with R⁶;

W is N or C(R¹²);

X is N or N-oxide;

Z is S, S(═O) or S(═O)₂;

R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹;

each R² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR²NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;

each R³ is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴R⁵, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;

each R⁴ and R⁵ is independently selected from the group consisting of H, alkyl, cycloalkyl, cycloalkylalkyl, heterocyclyl, heterocyclylalkyl, aryl, aralkyl, heteroaryl, heteroaralkyl, —OR⁷, —C(O)R⁷, and —C(O)OR⁷, wherein each of said alkyl, cycloalkyl, cycloalkylalkyl, heterocyclyl, heterocyclylalkyl, aryl, aralkyl, heteroaryl, or heteroaralkyl is optionally substituted with 1-4 R⁸ moieties;

or R⁴ and R⁵, when attached to the same nitrogen atom, are optionally taken together with the nitrogen atom to which they are attached to form a 3-6 membered heterocyclic ring having 0-2 additional heteroatoms selected from N, O or S;

each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;

each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;

each R⁸ is independently selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹, wherein each of said alkyl, cycloalkyl, heteroacyclyl, aryl, and heteroaryl is optionally independently substituted with 1-3 moieties selected from the group consisting of halo, alkyl, cycloalkyl, heterocyclyl, aryl, heteroaryl, —NO₂, —OR¹⁰, —(C₁-C₆ alkyl)-OR¹⁰, —CN,

—NR¹⁰OR¹¹, —C(O)OR¹⁰, —C(O)NR¹⁰OR¹¹, —CF³, —OCF₃, —NR¹⁰C(O)OR¹¹, and —NR¹⁰C(O)R⁴⁰;

or two R⁸ groups, when attached to the same carbon atom, are optionally taken together with the carbon atom to which they are attached to form a C═O or a C═S group;

each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;

each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;

each R¹¹ is independently H or alkyl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S;

each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴R⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties; and

R⁴⁰ is selected from the group consisting of cycloalkyl, heterocyclyl, aryl and heteroaryl, wherein each of said cycloalkyl, heterocyclyl, aryl and heteroaryl are optionally independently substituted with 1-3 moieties independently selected from the group consisting of —CN, —OH, halo, alkyl, haloalkyl, alkoxy, and —NR¹⁰R¹¹;

with the proviso that the compound of Formula I excludes any one of the following: 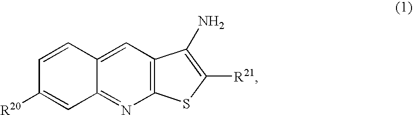
wherein R²⁰ is H, —CH₃ or —OCH₃ and R²¹ is —C(O)CH₃, —C(O)CH═CH-phenyl or —C(O)CH═CH-(4-methoxyphenyl); 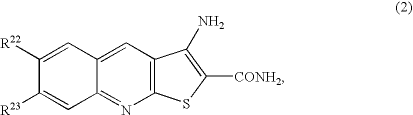
wherein R²² and R²³ are independently H or methoxy; 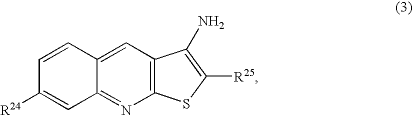
wherein R²⁴ is methyl, methoxy or —Cl and R²⁵ is —CONH₂ or —CO₂Et; 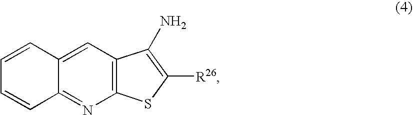
wherein R²⁶ is —CO₂Me, —CO₂Et, —CO₂H, —C(O)-phenyl, —C(O)-p-methylphenyl, —C(O)-p-bromophenyl, —C(O)CH₃, —CN, —C(O)NH-phenyl, —C(O)NH-p-methoxyphenyl, —C(O)NHNH₂, —C(O)NH-p-chlorophenyl, 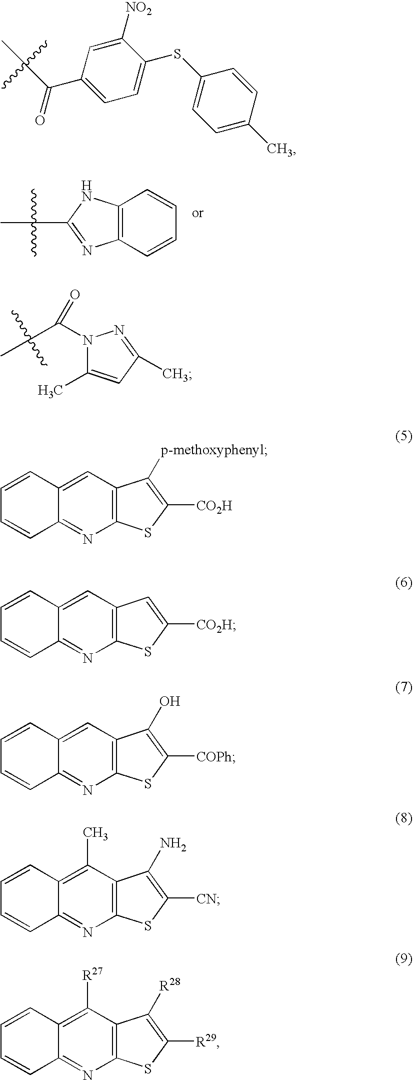
wherein:

R²⁷ is H, —OH, —OCH₃ or —OCH(CH₃)₂,

R²⁸ is —OH, —OCH₂CN or —OC(O)NH(CH₂)₅CN, and

R²⁹ is —C(O)OCH(CH₃)₂ or —C(O)O-cyclohexyl; 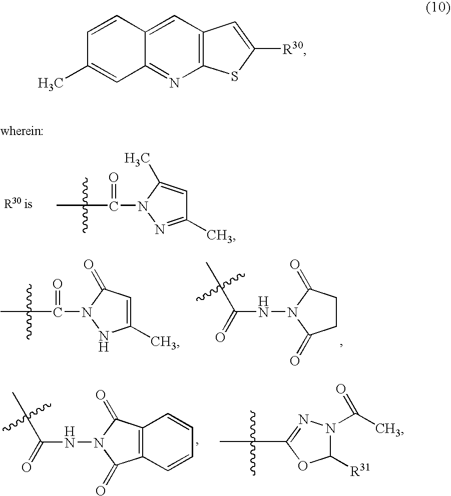
—CO₂CH₃, —CO₂C₂H₅, —C(O)NH₂, —C(O)NHNH₂, or —C(O)NHCH₃ and R³¹ is C₆H₅, p-OHC₆H₄ or p-CH₃C₆H₄; 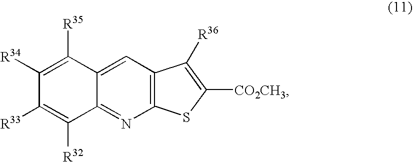
wherein:

R³² is H or NO₂,

R³³ and R³⁴ are independently H, —OCH₃ or —OC₂H₅,

R³⁵ is H or —OCH₃, and

R³⁶ is H, CH₃ or C₆H₅; 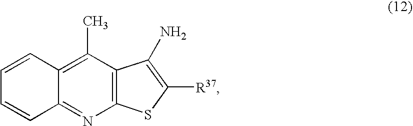
wherein:

R³⁷ is —CO₂Me, —CO₂Et, —CO₂H, —C(O)NH₂, —C(O)NHNH₂, —CN, —C(O)NH-p-methoxyphenyl, —C(O)NH-(2-pyridyl) or 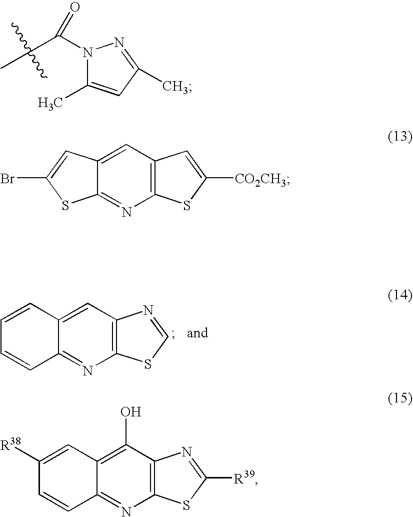
wherein R³⁸ is H, methyl or CF₃ and R³⁹ is SMe, SOMe, SO₂Me, Cl, NH(CH₂)NEt₂, or N-(N′-methyl)piperazinyl.

In another embodiment, the present invention provides a compound represented by the structural Formula I, or a pharmaceutically acceptable salt, solvate, or ester thereof, wherein in formula I,

ring Y is a 5- to 6-membered aryl or a 5- or 6-membered heteroaryl fused as shown in Formula I, wherein in said aryl and heteroaryl each substitutable ring carbon is independently substituted with R² and each substitutable ring nitrogen is independently substituted with R⁶;

W is N or C(R¹²);

X is N or N-oxide;

Z is S, S(═O) or S(═O)₂;

R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹;

each R² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₋OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;

each R³is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴R⁵, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;

each R⁴ and R⁵ is independently selected from the group consisting of H, alkyl, cycloalkyl, cycloalkylalkyl, heterocyclyl, heterocyclylalkyl, aryl, aralkyl, heteroaryl, heteroaralkyl, —OR⁷, —C(O)R⁷, and —C(O)OR⁷, wherein each of said alkyl, cycloalkyl, cycloalkylalkyl, heterocyclyl, heterocyclylalkyl, aryl, aralkyl, heteroaryl, or heteroaralkyl is optionally substituted with 14 R⁸ moieties;

or R⁴ and R⁵, when attached to the same nitrogen atom, are optionally taken together with the nitrogen atom to which they are attached to form a 3-6 membered heterocyclic ring having 0-2 additional heteroatoms selected from N, O or S;

each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;

each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 14 R⁸ moieties;

each R⁸ is independently selected from the group consisting of halo, alkyl, —OR¹⁰, —(C₁-C₆ alkyl)-OR¹⁰, —CN, —NR¹⁰R¹¹, —(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹;

or two R⁸ groups, when attached to the same carbon atom, are optionally taken together with the carbon atom to which they are attached to form a C═O or a C═S group;

each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;

each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;

each R¹¹ is independently H or alkyl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S; and

each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—R⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;

with the proviso that the compound of Formula I excludes any one of the following: 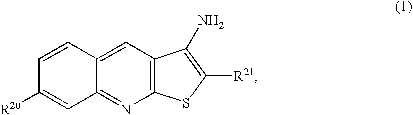
wherein R²⁰ is H, —CH₃ or —OCH₃ and R²¹ is —C(O)CH₃, —C(O)CH═CH-phenyl or —C(O)CH═CH-(4-methoxyphenyl); 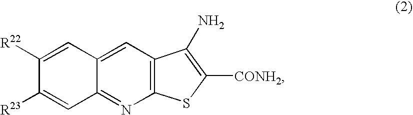
wherein R²² and R²³ are independently H or methoxy; 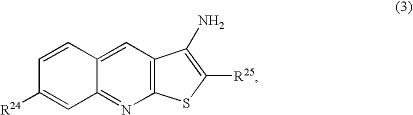
wherein R²⁴ is methyl, methoxy or —Cl and R²⁵ is —CONH₂ or —CO₂Et; 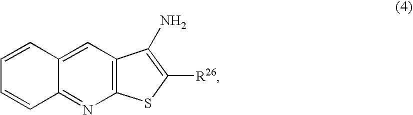
wherein R²⁶ is —CO₂Me, —CO₂Et, —CO₂H, —C(O)-phenyl, —C(O)-p-methylphenyl, —C(O)-p-bromophenyl, —C(O)CH₃, —CN, —C(O)NH-phenyl, —C(O)NH-p-methoxyphenyl, —C(O)NHNH₂, —C(O)NH-p-chlorophenyl, 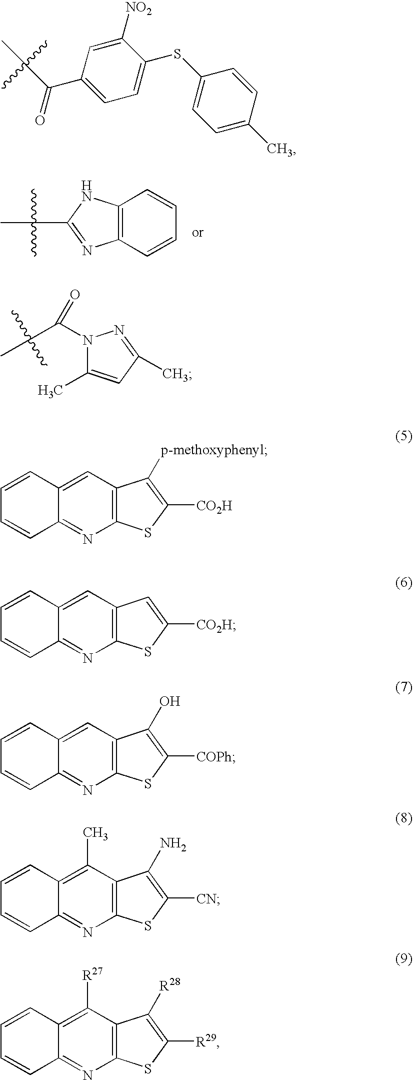
wherein:

R²⁷ is H, —OH, —OCH₃ or —OCH(CH₃)₂,

R²⁸ is —OH, —OCH₂CN or —OC(O)NH(CH₂)₅CN, and

R²⁹ is —C(O)OCH(CH₃)₂ or —C(O)O-cyclohexyl; 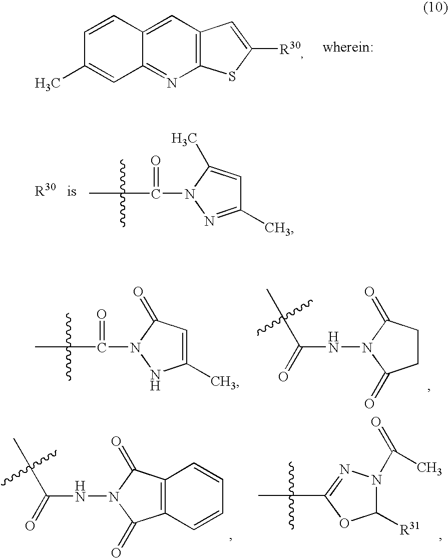
—CO₂CH₃, —CO₂C₂H₅, —C(O)NH₂, —C(O)NHNH₂, or —C(O)NHCH₃ and R³¹ is C₆H₅, p-OHC₆H₄ or p-CH₃C₆H₄; 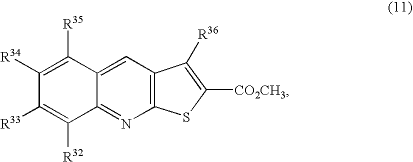
wherein:

R³² is H or NO₂,

R³³ and R³⁴ are independently H, —OCH₃ or —OC₂H₅,

R³⁵ is H or —OCH₃, and

R³⁶ is H, CH₃ or C₆H₅; 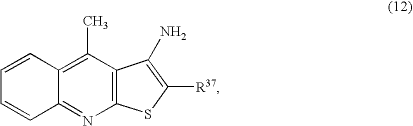
wherein:

R³⁷ is —CO₂Me, —CO₂Et, —CO₂H, —C(O)NH₂, —C(O)NHNH₂, —CN, —C(O)NH-p-methoxyphenyl, —C(O)NH-(2-pyridyl) or 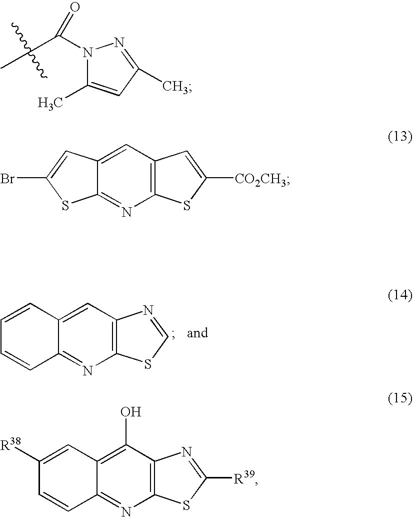
wherein R³⁸ is H, methyl or CF₃ and R³⁹ is SMe, SOMe, SO₂Me, Cl, NH(CH₂)NEt₂, or N-(N′-methyl)piperazinyl.

Pharmaceutical formulations or compositions for the treatment of cellular proliferative diseases, disorders associated with KSP kinesin activity and/or for inhibiting KSP kinesin activity in a subject comprising administering a therapeutically effective amount of at least one of the inventive compounds and a pharmaceutically acceptable carrier to the subject also are provided.

Methods of treating cellular proliferative diseases, disorders associated with KSP kinesin activity and/or for inhibiting KSP kinesin activity in a subject comprising administering to a subject in need of such treatment an effective amount of at least one of the inventive compounds also are provided.

Other than in the operating examples, or where otherwise indicated, all numbers expressing quantities of ingredients, reaction conditions, and so forth used in the specification and claims are to be understood as being modified in all instances by the term “about.”

### DETAILED DESCRIPTION

In one embodiment, the present invention discloses compounds represented by structural Formula I or a pharmaceutically acceptable salt, solvate or ester thereof, wherein the various moieties are as described above.

In one embodiment, the present invention discloses compounds represented by Formula II:

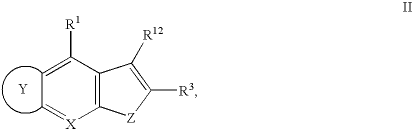

wherein ring Y, X, Z, R¹, R³ and R¹² are as defined above.

In one embodiment, the present invention discloses compounds represented by Formula III:

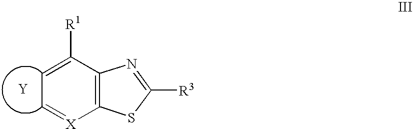

wherein ring Y, X, R¹, and R³ are as defined above.

In another embodiment, in formula I, II or III, X is N.

In another embodiment, in formula I, II or III, X is N-oxide.

In another embodiment, in formula I or II, Z is S.

In another embodiment, in formula I or II, Z is S(═O).

In another embodiment, in formula I or II, Z is S(═O)₂.

In another embodiment, ring Y in formula I, II or III is benzo wherein each substitutable ring carbon is independently substituted with R².

In another embodiment, wherein ring Y in formula I, II or III is benzo wherein each substitutable ring carbon is independently substituted with R², R² is H, alkyl, aryl, aralkyl, cycloalkyl, cycloalkylalkyl, —CF₃, alkylsilyl, alkoxy or —NR⁴R⁵.

In another embodiment, in formula I, II or III, R⁶ is H, alkyl, aralkyl, haloalkyl, cycloalkylalkyl or —C(O)OR⁷ wherein R⁷ is alkyl.

In another embodiment, in formula I or II, R¹² is H, halo, —NR⁴R⁵ or —OR⁷.

In another embodiment, in formula I, II or III, R³ is H, alkyl, heterocyclyl, heteroaryl, —(CR¹⁰R¹¹)₁₋₆—OR⁷, —C(O)R⁴, —C(O)OR⁷, —C(O)NR⁴R⁵, —C(S)NR⁴R⁵, —C(O)NR⁴OR⁷, —C(O)NR⁷NR⁴R⁵, —NR⁴R⁵, —NR⁴C(O)R⁵, —NR⁴C(O)NR⁴R⁵, —(CR¹⁰R¹¹)₀₋₆SR⁷, S(O₂)R⁷, —S(O₂)NR⁴R⁵, —CN, —C(═NR⁷)NR⁴, —C(O)NR⁷(CH₂)₁₋₁₀NR⁴R⁵, or —C(O)NR⁷(CH₂)₁₋₁₀OR⁷, wherein said alkyl, heterocyclyl or heteroaryl is optionally substituted with 1-3 R⁹ moieties.

In another embodiment, in formula I, II, or III, R¹ is H, halo, —S-alkyl, alkoxy or hydroxy.

In another embodiment, in formula I, II, or III, R¹ is H, Cl, OH or —SCH₃.

In another embodiment, the present invention discloses compounds represented by Formula II-a: 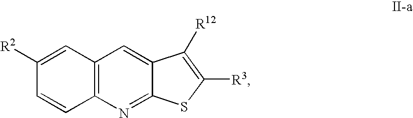
wherein R², R³, and R¹² are as set forth for formula I or II.

In another embodiment, the present compounds are represented by Formula II-a, wherein:
R² is alkyl, aryl, aralkyl, cycloalkyl, cycloalkylalkyl, —CF₃, alkylsilyl, or —NR⁴R⁵;
R³ is H, heterocyclyl, heteroaryl, —C(O)OR⁷, —C(O)R⁴, —C(O)NR⁴R⁵, —C(S)N R⁴R⁵, —C(O)N(R⁴)OR⁷, —NR⁴R⁵, —NR⁴C(O)R⁵, —NR⁴C(O)NR⁴R⁵, —SO₂R⁷, —SO₂NR⁴R⁵, —CN, —(CR¹⁰R¹¹)₁₋₆SR⁷, or —C(═NR⁷)NR⁴R⁵; and
R¹² is H, halo, —NR⁴R⁵, or —OR⁷.

In another embodiment, the present compounds are represented by Formula II-a, wherein:
R² is alkyl or alkylsilyl; wherein said alkyl is C₁-C₆ alkyl and said alkylysilyl is C₁-C₆ alkylsilyl;
R³ is —CN, —C(O)NR⁴R⁵, —C(O)R⁴, —C(S)NR⁴R⁵, —C(═NR⁷)NR⁴R⁵, heterocyclyl, —C(O)OR⁷, —C(O)N(R⁴)OR⁷, —SO₂R⁷, —SO₂NR⁴R⁵, —N(R⁴)C(O)R⁵, or —N(R⁴)C(O)NR⁴R⁵; wherein said —C(O)NR⁴R⁵ is —C(O)N(R⁶¹)₂, said —C(O)R⁴ is —C(O)R⁶², said —C(S)NR⁴R⁵ is —C(S)N(R⁶⁰)₂, said —C(═NR⁷)NR⁴R⁵ is —C(═NR⁶ )N(R⁶⁰)₂, said heterocyclyl is tetrazolyl, said —C(O)OR⁷ is —C(O)OR⁶¹, said —C(O)N(R⁴)OR⁷ is —C(O)N(R⁶⁰)OR⁶⁰, said —SO₂R⁷ is —SO₂R⁶⁰, said —SO₂NR⁴R⁵ is —SO₂N(R⁶⁰)₂, said —N(R⁴)C(O)R⁵ is —N(R⁶⁰)C(O)R⁶⁰, and said —N(R⁴)C(O)NR⁴R⁵ is —N(R⁶⁰)C(O)N (R⁶⁰)₂;
R¹² is H, halo, —NR⁴R⁵, or —OR⁷; wherein said —NR⁴R⁵ is —N(R⁶⁰)₂, and said —OR⁷ is —OR⁶⁰;
each R⁶⁰ independently is H or C₁-C₆ alkyl;
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰; and
R⁶² is N-pyrrolidinyl, N-piperidinyl, N-piperazinyl, N,N′-methylpiperazinyl; wherein each member of R⁶² is optionally substituted with —OR⁶⁰, —CO₂R⁶⁰, or —N(R⁶⁰)₂; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

In another embodiment, the present compounds are represented by Formula II-a, wherein:
R² is alkyl or alkylsilyl; wherein said alkyl is C₁-C₆ alkyl and said alkylysilyl is C₁-C₆ alkylsilyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

In another embodiment, the present compounds are represented by Formula II-a, wherein:
R² is C₁-C₆ alkylsilyl;
R³ is —C(O)NR⁴R⁵ wherein said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

In another embodiment, the present compounds are represented by Formula II-a, wherein:
R² is C₁-C₆ alkyl; and
R³ is —CN, —C(O)N(R⁶¹)₂ or —C(O)OR⁶¹; wherein said —C(O)N(R⁶¹)₂ is —C(O)N(R⁶³)₂, and said —C(O)OR⁶¹ is —C(O)OR⁶⁰; and
R⁶³ is H, C₁-C₆ alkyl or phenyl, wherein said C₁-C₆ alkyl is optionally substituted with —N(R⁶⁰)C(O)R⁶⁰ or —N(R⁶⁰)₂, and said phenyl is is optionally substituted with 1-2 moieties independently selected from the group consisting of —N(R⁶⁰)₂ and —N(R⁶⁰)C(O)R⁷⁰.

In another embodiment, for each of the above embodiments, wherein the compound is represented by formula IIa, R¹² is H.

In another embodiment, the present compounds are represented by Formula II-a, wherein:
R² is alkyl;
R³ is —C(O)NR⁴R⁵;
R⁴ and R⁵ are independently selected from the group consisting of H and alkyl, wherein said alkyl is optionally substituted with 1-4 R⁸ moieties; each R⁸ is independently selected from the group consisting of —NR¹⁰R¹¹ and aryl; wherein said aryl is optionally substituted with 1-3 moieties independently selected from the group consisting of alkyl, —NR¹⁰R¹¹ and —NR¹⁰C(O)R⁴⁰;
each R¹⁰ is independently H or alkyl;
each R¹¹ is independently H or alkyl;
R¹² is H; and
R⁴⁰ is selected from the group consisting of aryl and heteroaryl, wherein said aryl and heteroaryl are optionally independently substituted with 1-3 moieties independently selected from the group consisting of —CN, —OH, halo, alkyl, haloalkyl, alkoxy, and —NR¹⁰R¹¹.

In another embodiment, the present compounds are represented by Formula II-a, wherein:
R²is alkyl;
R³is —C(O)NR⁴R⁵;
R⁴ and R⁵ are independently selected from the group consisting of H and alkyl, wherein said alkyl is optionally substituted with 14 R⁸ moieties;
each R⁸ is independently selected from the group consisting of —NR¹⁰R¹¹ and aryl; wherein said aryl is optionally substituted with 1-3 moieties independently selected from the group consisting of alkyl, —NR¹⁰R¹¹ and —NR¹⁰C(O)R⁴⁰; wherein said R⁸ aryl is phenyl;
each R¹⁰ is independently H or alkyl;
each R¹¹ is independently H or alkyl;
R¹² is H; and
R⁴⁰ is selected from the group consisting of aryl and heteroaryl, wherein said aryl and heteroaryl are optionally independently substituted with 1-3 moieties independently selected from the group consisting of —CN, —OH, halo, alkyl, haloalkyl, alkoxy, and —NR¹⁰R¹¹.

In another embodiment, the present compounds are represented by Formula II-a, wherein:
R² is alkyl;
R³ is —C(O)NR⁴R⁵;
R⁴ and R⁵ are independently selected from the group consisting of H and alkyl, wherein said alkyl is optionally substituted with 14 R⁸ moieties;
each R⁸ is independently selected from the group consisting of —NR¹⁰R¹¹ and aryl; wherein said aryl is optionally substituted with 1-3 moieties independently selected from the group consisting of alkyl, —NR¹⁰R¹¹ and —NR¹⁰C(O)R⁴⁰;
each R¹⁰ is independently H or alkyl;
each R¹¹ is independently H or alkyl;
R¹² is H; and
R⁴⁰ is selected from the group consisting of aryl and heteroaryl, wherein said aryl and heteroaryl are optionally independently substituted with 1-3 moieties independently selected from the group consisting of —CN, —OH, halo, alkyl, haloalkyl, alkoxy, and —NR¹⁰R¹¹; wherein said R⁴⁰ heteroaryl is selected from the group consisting of furanyl, pyrazolyl, pyrazinyl, oxazolyl, and isoxazolyl, each of which is optionally substituted.

In another embodiment, the present compounds of formula I or II are represented by Formula II-b: 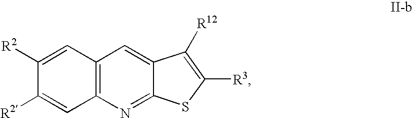
wherein R^{2′} is selected from the members of R², wherein R^{2′} and R² can be the same of different; and R³ and R¹² are as set forth for formula I or II.

In another embodiment, the present compounds are represented by the formula IIb, wherein:
R² is alkyl, aryl, aralkyl, cycloalkyl, cycloalkylalkyl, —CF₃, alkylsilyl, or —NR⁴R⁵;
R² is alkyl, aryl, aralkyl, cycloalkyl, cycloalkylalkyl, —CF₃, alkylsilyl, or —NR⁴R⁵;
R³ is H, heterocyclyl, heteroaryl, —C(O)R⁴, —C(O)OR⁷, —C(O)NR⁴R⁵, —C(S)NR⁴R⁵, —C(O)N(R⁴)OR⁷, —NR⁴R⁵, —N(R⁴)C(O)R⁵, —N(R⁴)C(O)NR⁴R⁵, —SO₂R⁷, —SO₂NR⁴R⁵, —CN, —(CR¹⁰R¹¹)₁₋₆SR⁷, or —C(═NR⁷)NR⁴R⁵; and
R¹² is H, halo, —NR⁴R⁵, or —OR⁷.

In another embodiment, the present compounds are represented by the formula IIb, wherein:
R² is alkyl or alkylsilyl; wherein said alkyl is C₁-C₆ alkyl, and said alkylsilyl is C₁-C₆ alkylsilyl;
R² is alkyl or alkylsilyl; wherein said alkyl is C₁-C₆ alkyl, and said alkylsilyl is C₁-C₆ alkylsilyl;
R³ is —CN, —C(O)NR⁴R⁵, —C(O)R⁴, —C(S)NR⁴R⁵, —C(═NR⁷)NR⁴R⁵, heterocyclyl, —C(O)OR⁷, —C(O)N(R⁴)OR⁷, —SO₂R⁷, —SO₂NR⁴R⁵, —N(R⁴)C(O)R⁵, or —N(R⁴)C(O)NR⁴R⁵; wherein said —C(O)NR⁴R⁵ is —C(O)N(R⁶¹)₂, said —C(O)R⁴ is
—C(O)R⁶², said —C(S)NR⁴R⁵ is —C(S)N(R⁶⁰)₂, said —C(═NR⁷)NR⁴R⁵ is
—C(═NR⁶⁰)N(R⁶⁰)₂, said heterocyclyl is tetrazolyl, said —C(O)OR⁷ is —C(O)OR⁶¹, said —C(O)N(R⁴)OR⁷ is —C(O)N(R⁶⁰)OR⁶⁰, said —SO₂R⁷ is —SO₂R⁶⁰, said —SO₂NR⁴R⁵ is —SO₂N(R⁶⁰)₂, said —N(R⁴)C(O)R⁵ is —N(R⁶⁰)C(O)R⁶⁰, and said —N(R⁴)C(O)NR⁴R⁵ is —N(R⁶⁰)C(O)N(R⁶⁰)₂;
R¹² is H, halo, —NR⁴R⁵, or —OR⁷; wherein said —NR⁴R⁵ is —N(R⁶⁰)₂, and said —OR⁷ is —OR⁶⁰;
each R⁶⁰ independently is H or C₁-C₆ alkyl;
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
R⁶² is N-pyrrolidinyl, N-piperidinyl, N-piperazinyl, N,N′-methylpiperazinyl; wherein each member of R⁶² is optionally substituted with —OR⁶⁰, —CO₂R⁶⁰, or —N(R⁶⁰)₂; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

In another embodiment, the present compounds are represented by the formula IIb as set forth in each of the above embodiments of formula IIb set forth in the preceding paragraphs, and wherein the alkylsilyl group in said R² and R³ is (C₁-C₆ alkyl)₃silyl.

In another embodiment, the present compounds are represented by the formula IIb as set forth in each of the above embodiments of formula IIb set forth in the preceding paragraphs, and wherein R¹² is H.

In another embodiment, the present compounds are represented by the formula IIb, wherein the 5- to 6-membered heterocyclyl in R⁶¹ is morpholinyl, piperidinyl, pyrrolidinyl, or piperazinyl.

In another embodiment, the present compounds are represented by the formula IIb, wherein:
R² and R^{2′} are independently alkyl; wherein said alkyl is C₁-C₆ alkyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

In another embodiment, the present compounds are represented by the formula IIb, wherein:
R² and R^{2′} are independently C₁-C₆ alkylsilyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); ); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

In another embodiment, the present compounds are represented by any one of formula I, II, or IIa, wherein:
R² is alkyl, said alkyl being t-butyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

In another embodiment, the present compounds are represented by any one of formula I, II, or IIa, including any of the above-mentioned embodiments of said formulae I, II, or IIa, wherein R¹² is H.

In another embodiment, the present compounds are represented by formula IIa, wherein:
R² is alkyl, said alkyl being t-butyl or i-propyl;
R^{2′} is alkyl, said alkyl being methyl or ethyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

In another embodiment, the present compounds are represented by formula IIa, wherein:
R³ is —CN, —C(O)OR⁶¹ or —C(O)NR⁴R⁵; wherein said —C(O)OR⁶¹ is —C(O)OR⁶⁰, and said —C(O)NR⁴R⁵ is —C(O)N(R⁶³)₂; and
each R⁶³ independently is H or C₁-C₆ alkyl wherein said C₁-C₆ alkyl of said R⁶³ is optionally substituted with —N(R⁶⁰)C(O)R⁶⁰ or —N(R⁶⁰)₂; wherein each R⁶⁰ independently is H or C₁-C₆ alkyl.

In another embodiment, the present compounds are represented by formula IIa or IIb, wherein R¹² is H.

In another embodiment, the present compounds are represented by Formula III-a:

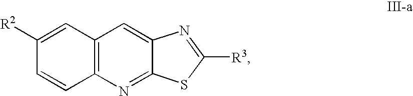

wherein:
R² is alkyl, aryl, aralkyl, cycloalkyl, cycloalkylalkyl, —CF₃, alkylsilyl, alkoxy or —NR⁴R⁵; and
R³ is H, heterocyclyl, heteroaryl, —C(O)OR⁷, —C(O)NR⁴R⁵, —C(S)NR⁴R⁵, —C(O)NR⁴OR⁷, —NR⁴R⁵, —N(R⁴)C(O)R⁵, —N(R⁴)C(O)NR⁴R⁵, —SO₂R⁷, —SO₂NR⁴R⁵, —CN, —(CR¹⁰R¹¹)₀₋₆SR⁷, or —C(═NR⁷)NR⁴R⁵.

In another embodiment, the present compounds are represented by Formula III-a, wherein R³ is —C(O)OR⁷, —C(O)NR⁴R⁵, —NR⁴R⁵, —NR⁴C(O)R⁵, —NR⁴C(O)NR⁴R⁵, —(CR¹⁰R¹¹)₀₋₆SR⁷, or —CN.

In another embodiment, the present compounds are represented by Formula III-a, wherein:
R² is alkyl; wherein said alkyl is C₁-C₆ alkyl;
R³ is —CN, —C(O)OR⁷, —(CR¹⁰R¹¹)₀₋₆SR⁷, —C(O)NR⁴R⁵, —N(R⁴)C(O)NR⁴R⁵, —NR⁴R⁵, and —N(R⁴)C(O)R⁵; wherein said —C(O)OR⁷ is —C(O)OR⁶⁰, said ‘(CR¹⁰R¹¹)₀₋₆SR⁷ is —SR⁶⁰, said —C(O)NR⁴R⁵ is C(O)N(R⁶⁰)₂, said —N(R⁴)C(O)NR⁴R⁵is —NR⁶⁰C(O)N(R⁶⁰)₂, said —NR⁴R⁵ is —N(R⁶⁰)₂, and said —N(R⁴)C(O)R⁵ is —NR⁶⁰C(O)R⁶⁰; and

each R⁶⁰ is H or C₁-C₆ alkyl.

In another embodiment, the present compounds are represented by Formula III-a, wherein:
R² is alkyl or alkylsilyl; wherein said alkyl is C₁-C₆ alkyl, and said alkylsilyl is C₁-C₆ alkylsilyl;
R³ is —CN, —C(O)OR⁷, —C(O)R⁷, —C(O)NR⁴R⁵, —C(S)NR⁴R⁵, —C(═NR⁷)NR⁴R⁵, heterocyclyl, —C(O)N(R⁴)OR⁷, —SO₂R⁷, S(O)₁₋₂NR⁴R⁵, —NR⁴C(O)R⁵ or —NR⁴C(O)NR⁴R⁵; wherein said —C(O)OR⁷ is —C(O)OR⁶¹, said

—C(O)R⁷ is —C(O)R⁶², said —C(O)NR⁴R⁵ is —C(O)N(R⁶¹)₂, said —C(S)NR⁴R⁵ is —C(S)N(R⁶⁰)₂, said —C(═NR⁷)NR⁴R⁵ is —C(═NR⁶⁰)N(R⁶⁰)₂, said heterocyclic is tetrazolyl, said —C(O)N(R⁴)OR⁷ is —C(O)N(R⁶⁰)OR⁶⁰, said —SO₂R⁷ is —SO₂R⁶⁰, said S(O)₁₋₂NR⁴R⁵ is —SO₂N(R⁶⁰)₂, said —NR⁴C(O)R⁵ is —N(R⁶⁰)C(O)R⁶⁰, and said —NR⁴C(O)NR⁴R⁵ is —N(R⁶⁰)C(O)N(R⁶⁰)₂;
each R⁶⁰ independently is H or C₁-C₆ alkyl;
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
R⁶² is N-pyrrolidinyl, N-piperidinyl, N-piperazinyl, N,N′-methylpiperazinyl; wherein each member of R⁶² is optionally substituted with —OR⁶⁰, —CO₂R⁶⁰, or —N(R⁶⁰)₂; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

In another embodiment, the present compounds are represented by Formula III-a, wherein:
R² is alkyl; wherein said alkyl is C₁-C₆ alkyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

In another embodiment, the present compounds are represented by Formula III-a, wherein:
R² is C₁-C₆ alkylsilyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

In another embodiment, the present compounds are represented by Formula III-b:

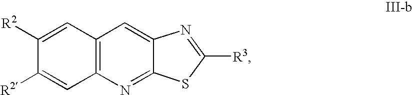

wherein:
R² is alkyl, aryl, aralkyl, cycloalkyl, cycloalkylalkyl, —CF₃, alkylsilyl, alkoxy or —NR⁴R⁵;
R² is alkyl, aryl, aralkyl, cycloalkyl, cycloalkylalkyl, —CF₃, alkylsilyl, alkoxy or —NR⁴R⁵; and
R³ is H, heterocyclyl, heteroaryl, —C(O)OR⁷, —C(O)NR⁴R⁵, —C(S)NR⁴R⁵, —C(O)NR⁴OR⁷, —NR⁴R⁵, —NR⁴C(O)R⁵, —NR⁴C(O)NR⁴R⁵, —SO₂R⁷, —SO₂NR⁴R⁵, —CN, —(CR¹⁰R¹¹)₀₋₆SR⁷, or —C(═NR⁷)NR⁴R⁵.

In another embodiment, the present compounds are represented by Formula III-b, wherein R³ is —C(O)NR⁴R⁵, —NR⁴R⁵, —NR⁴C(O)R⁵, —NR⁴C(O)NR⁴R⁵, —(CR¹⁰R¹¹)₀₋₆SR⁷, or —CN.

In another embodiment, the present compounds are represented by Formula III-b, wherein:
R² and R^{2′} are independently alkyl; wherein said alkyl is C₁-C₆-alkyl;
R³ is —CN, —(CR¹⁰R¹¹)₀₋₆SR⁷, —C(O)NR⁴R⁵, —NR⁴C(O)NR⁴R⁵, —NR⁴R⁵, or —NR⁴C(O)R⁵; wherein said —(CR¹⁰R¹¹)₀₋₆SR⁷ is —SR⁶⁰, said —C(O)NR⁴R⁵ is —C(O)N(R⁶⁰)₂, said —NR⁴C(O)NR⁴R⁵ is —NR⁶⁰C(O)N(R⁶⁰)₂, said —NR⁴R⁵ is —N(R⁶⁰)₂, and said —NR⁴C(O)R⁵ is —NR⁶⁰C(O)R⁶⁰; and
each R⁶⁰ independently is H or C₁-C₆ alkyl.

In another embodiment, the present compounds are represented by Formula III-b, wherein:
R² is alkyl or alkylsilyl; wherein said alkyl is C₁-C₆ alkyl and said alkylsilyl is C₁-C₆ alkylsilyl;
R^{2′} is alkyl or alkylsilyl; wherein said alkyl is C₁-C₆ alkyl and said alkylsilyl is C₁-C₆ alkylsilyl;
R³ is —CN, —C(O)OR⁷, —C(O)R⁷, —C(O)NR⁴R⁵, —C(S)NR⁴R⁵, —C(═NR⁷)NR⁴R⁵, heterocyclyl, —C(O)N(R⁴)OR⁷, —SO₂R⁷, S(O)₁₋₂NR⁴R⁵, —NR⁴C(O)R⁵ or —NR⁴C(O)NR⁴R⁵; wherein said —C(O)OR⁷ is —C(O)OR⁶¹, said

—C(O)R⁷ is —C(O)R⁶², said —C(O)NR⁴R⁵ is —C(O)N(R⁶¹)₂, said —C(S)NR⁴R⁵ is —C(S)N(R⁶⁰)₂, said —C(═NR⁷)NR⁴R⁵ is —C(═NR⁶⁰)N(R⁶⁰)₂, said heterocyclic is tetrazolyl, said —C(O)N(R⁴)OR⁷ is —C(O)N(R⁶⁰)OR⁶⁰, said —SO₂R⁷ is —SO₂R⁶⁰, said S(O)₁₋₂NR⁴R⁵ is —SO₂N(R⁶⁰)₂, said —NR⁴C(O)R⁵ is —N(R⁶⁰)C(O)R⁶⁰, and said —NR⁴C(O)NR⁴R⁵ is —N(R⁶⁰)C(O)N(R⁶⁰)₂;
each R⁶⁰ independently is H or C₁-C₆ alkyl;
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
R⁶² is N-pyrrolidinyl, N-piperidinyl, N-piperazinyl, N,N′-methylpiperazinyl; wherein each member of R⁶² is optionally substituted with —OR⁶⁰, —CO₂R⁶⁰, or —N(R⁶⁰)₂; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

In another embodiment, the present compounds are represented by Formula III-b, wherein:
R² and R^{2′} are independently alkyl; wherein said alkyl is C₁-C₆ alkyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.
In another embodiment, the present compounds are represented by Formula III-b, wherein the 5- to 6-membered heterocyclyl in R⁶¹ is morpholinyl, piperidinyl, pyrrolidinyl, or piperazinyl.

In another embodiment, the present compounds are represented by Formula III-b, wherein:
R² and R^{2′} are independently C₁-C₆ alkylsilyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and each R⁶¹ independently is H, C₁-C₆ alkyl, phenyl, benzyl, morpholinyl, a 4-6 member β-lactam ring, or cyclopentyl, wherein said cyclopentyl is optionally substituted with —OR⁶⁰ and said C₁-C₆ alkyl is optionally substituted with —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

Representative compounds of the present invention include: 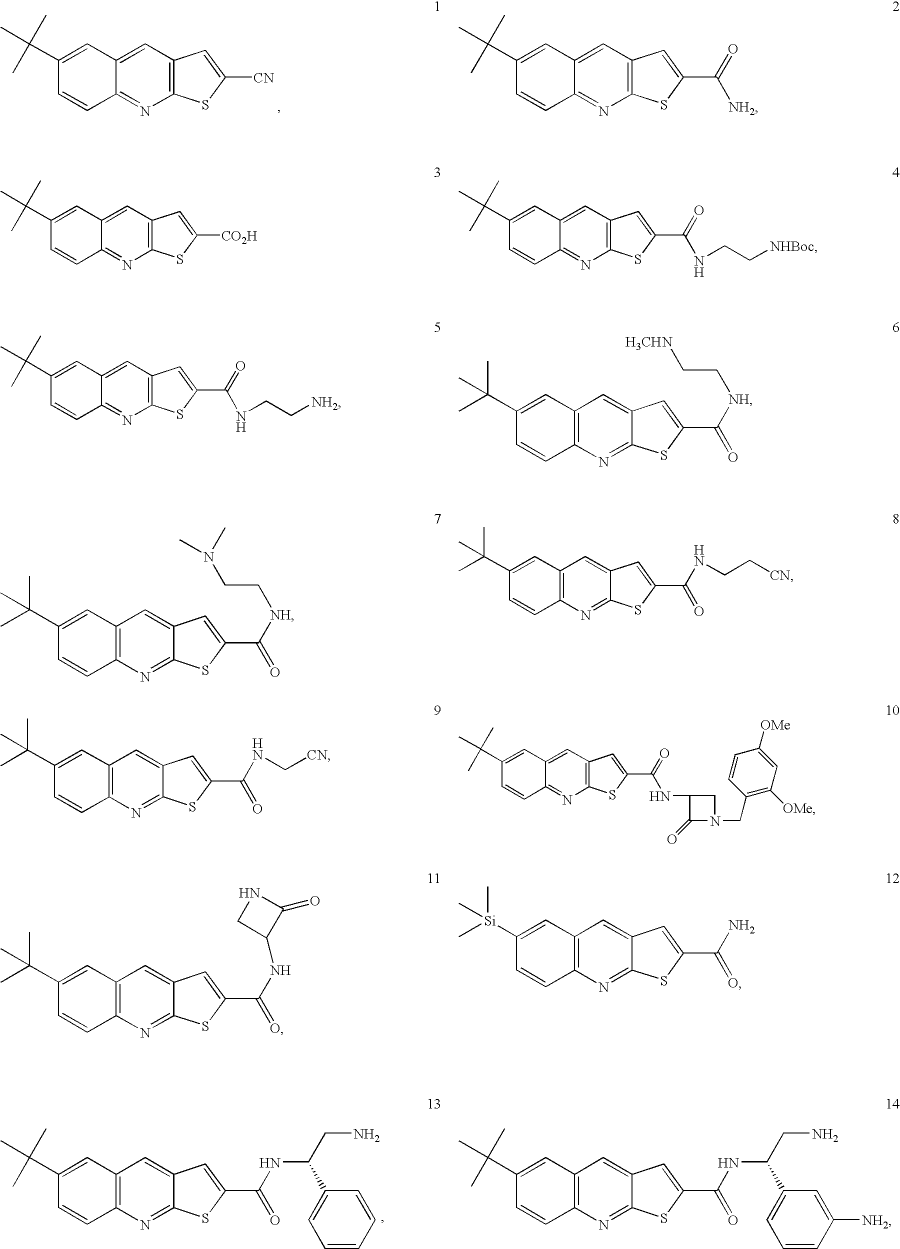 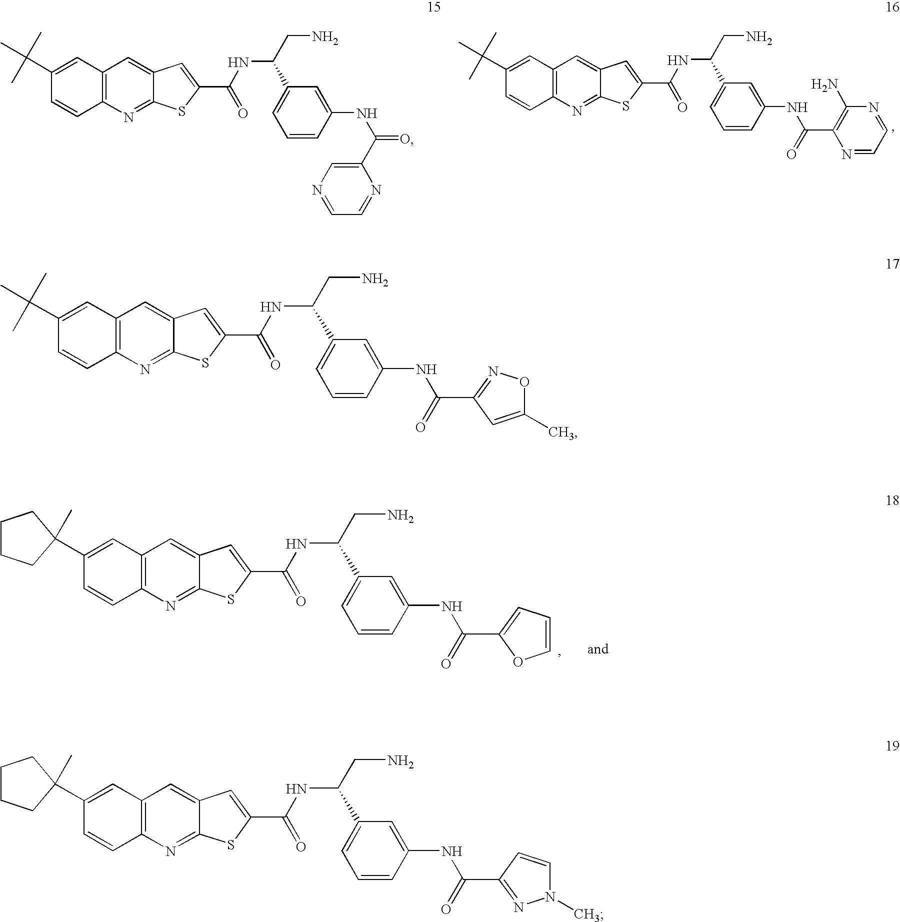
or a pharmaceutically acceptable salt or solvate thereof.

In another embodiment, the compounds are selected from the group consisting of 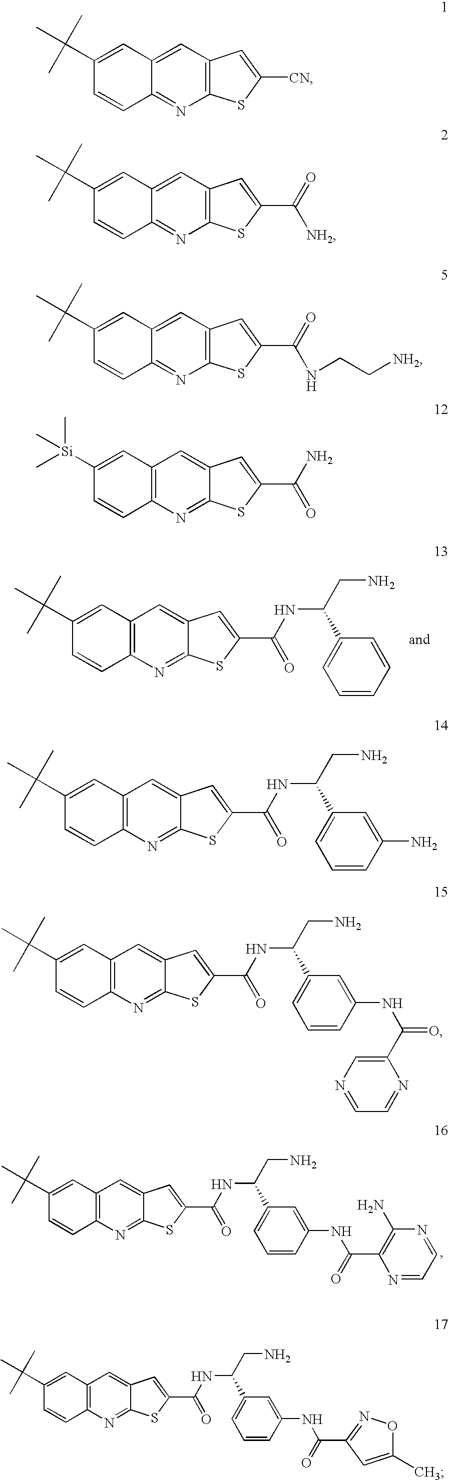
or a pharmaceutically acceptable salt or solvate thereof.

In other embodiments, the present invention provides processes for producing such compounds, pharmaceutical formulations or compositions comprising one or more of such compounds, and methods of treating or preventing one or more conditions or diseases associated with KSP kinesin activity such as those discussed in detail below.

As used above, and throughout the specification, the following terms, unless otherwise indicated, shall be understood to have the following meanings:

“Subject” includes both mammals and non-mammalian animals.

“Mammal” includes humans and other mammalian animals.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties. It should be noted that any atom with unsatisfied valences in the text, schemes, examples and tables herein is assumed to have the hydrogen atom(s) to satisfy the valences.

The following definitions apply regardless of whether a term is used by itself or in combination with other terms, unless otherwise indicated. Therefore, the definition of “alkyl” applies to “alkyl” as well as the “alkyl” portions of “hydroxyalkyl”, “haloalkyl”, “alkoxy”, etc.

As used herein, the term “alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. The alkyl group may be substituted with one or more substituents independently selected from the group consisting of halo, alkyl, aryl, cycloalkyl, cyano, hydroxy, alkoxy, amino, —NH(alkyl), —NH(cycloalkyl), —N(alkyl)₂, carboxy, —C(O)O-alkyl and —S(alkyl), wherein said alkyl, cycloalkyl and aryl are unsubstituted. Non-limiting examples of suitable alkyl groups include methyl, ethyl, n-propyl, isopropyl, n-butyl, t-butyl, n-pentyl, heptyl, nonyl, decyl, fluoromethyl, trifluoromethyl and cyclopropylmethyl. Unless otherwise stated, the term “alkyl” includes “alkenyl” and “alkynyl” as defined below.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. The alkenyl group may be substituted with one or more substituents independently selected from the group consisting of halo, alkyl, aryl, cycloalkyl, cyano, alkoxy and -S(alkyl), wherein said alkyl, cycloalkyl and aryl are unsubstituted. Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl, 3-methylbutynyl, n-pentynyl, and decynyl. The alkynyl group may be substituted with one or more substituents being independently selected from the group consisting of alkyl, aryl and cycloalkyl, wherein said alkyl, cycloalkyl and aryl are unsubstituted.

“Alkoxy” means an alkyl-O-group in which the alkyl group is as previously described. Useful alkoxy groups can comprise 1 to about 12 carbon atoms, preferably 1 to about 6 carbon atoms. Non-limiting examples of suitable alkoxy groups include methoxy, ethoxy and isopropoxy. The alkyl group of the alkoxy is linked to an adjacent moiety through the ether oxygen.

“Aryl” means an aromatic monocyclic or multicyclic ring system comprising about 5 to about 14 carbon atoms, preferably about 6 to about 10 carbon atoms. The aryl group can be substituted with one or more “ring system substituents” which may be the same or different, and are as defined herein. Non-limiting examples of suitable aryl groups include phenyl and naphthyl. Also included within the scope of the term “aryl”, as used herein, is a group in which an aromatic hydrocarbon ring is fused to one or more non-aromatic carbocyclic or heteroatom-containing rings, such as in an indanyl, phenanthridinyl or tetrahydronaphthyl, where the radical or point of attachment is on the aromatic hydrocarbon ring.

“Aralkyl” or “arylalkyl” means an alkyl group substituted with an aryl group in which the aryl and alkyl are as previously described. Preferred aralkyls comprise a lower alkyl group. Non-limiting examples of suitable aralkyl groups include benzyl, phenethyl and naphthlenylmethyl. The aralkyl is linked to an adjacent moiety through the alkylene group.

“Cycloalkyl” means a non-aromatic mono- or multicyclic hydrocarbon ring system comprising about 3 to about 12 carbon atoms, preferably about 5 to about 10 carbon atoms. A cycloalkyl may be fully saturated or may contain one or more units of unsaturation but is not aromatic. Preferred cycloalkyl rings contain about 5 to about 7 ring atoms. The cycloalkyl can be substituted with one or more “ring system substituents” which may be the same or different, and are as defined below. Non-limiting examples of suitable monocyclic cycloalkyls include cyclopropyl, cyclobutyl, cyclopentyl, cyclohexyl, cyclopentenyl, cyclohexenyl, cyclohepta-1,3-dienyl, and the like. Non-limiting examples of suitable multicyclic cycloalkyls include 1-decalinyl, norbornyl, adamantly, norbornylenyl and the like. The term “cycloalkyl” also includes hydrocarbon rings that are fused to one or more aromatic rings where the radical or point of attachment is on the non-aromatic ring.

“Halo” refers to fluorine, chlorine, bromine or iodine radicals.

“Heteroaryl” means a monocyclic or multicyclic aromatic ring system of about 5 to about 14 ring atoms, preferably about 5 to about 10 ring atoms, in which one or more of the atoms in the ring system is/are atoms other than carbon, for example nitrogen, oxygen or sulfur. Preferred heteroaryls contain about 5 to about 6 ring atoms. The “heteroaryl” can be optionally substituted with one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be oxidized to form the corresponding N-oxide. All regioisomers are contemplated, e.g., 2-pyridyl, 3-pyridyl and 4-pyridyl. Examples of useful 6-membered heteroaryl groups include pyridyl, pyrimidinyl, pyrazinyl, pyridazinyl and the like and the N-oxides thereof. Examples of useful 5-membered heteroaryl rings include furyl, thienyl, pyrrolyl, thiazolyl, isothiazolyl, imidazolyl, pyrazolyl and isoxazolyl. Useful bicyclic groups are benzo-fused ring systems derived from the heteroaryl groups named above, e.g., quinolyl, phthalazinyl, quinazolinyl, benzofuranyl, benzothienyl and indolyl. Also included within the scope of the term “heteroaryl” is a group in which a heteroaromatic ring is fused to one or more aromatic or non-aromatic rings where the radical or point of attachment is on the heteroaromatic ring.

“Heteroarylalkyl” or “heteroaralkyl” means an alkyl group substituted with a heteroaryl group in which the heteroaryl and alkyl are as previously described. Preferred heteroaralkyls contain a lower alkyl group. Non-limiting examples of suitable heteroaralkyl groups include pyridylmethyl, 2-(furan-3-yl)ethyl and quinolin-3-ylmethyl. The bond to the parent moiety is through the alkyl. “Heteroarylalkoxy” means a heteroaryl-alkyl-O-group in which the heteroaryl and alkyl are as previously described.

“Heterocyclyl” means a non-aromatic monocyclic or multicyclic ring system comprising about 3 to about 12 ring atoms, preferably about 5 to about 10 ring atoms, in which one or more of the atoms in the ring system is an element other than carbon, for example nitrogen, oxygen or sulfur, or combinations thereof. Preferred heterocyclyls contain about 5 to about 6 ring atoms. The prefix aza, oxa or thia before the heterocyclyl root name means that at least a nitrogen, oxygen or sulfur atom respectively is present as a ring atom. The heterocyclyl can be optionally substituted with one or more “ring system substituents” which may be the same or different, and are as defined herein. The nitrogen or sulfur atom of the heterocyclyl can be optionally oxidized to the corresponding N-oxide, S-oxide or S-dioxide. Non-limiting examples of suitable monocyclic heterocyclyl rings include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like. A heterocyclic ring may be fully saturated or may contain one or more units of unsaturation but is not aromatic. Suitable examples include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclyl” may also mean a single moiety (e.g., carbonyl) which simultaneously replaces two available hydrogens on the same carbon atom on a ring system. Examples of such moiety are pyrrolidone:

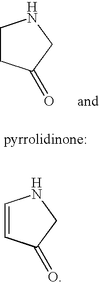

“Heterocyclylalkyl” means an alkyl group substituted with a heterocyclyl group in which the heterocyclyl and alkyl groups are as previously described. Preferred heterocyclylalkyls contain a lower alkyl group. The bond to the parent moiety is through the alkyl.

“Ring system substituent” means a substituent attached to an aromatic or non-aromatic ring system that, for example, replaces an available hydrogen on the ring system. Ring system substituents may be the same or different, each being independently selected from the group consisting of aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, heterocyclyl, Y₁Y₂N—, Y₁Y₂N-alkyl-, Y₁Y₂NC(O)— and Y₁Y₂NSO₂—, wherein Y₁ and Y₂ may be the same or different and are independently selected from the group consisting of hydrogen, alkyl, aryl, and aralkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Alkylamino” means an —NH₂ or —NH₃⁺ group in which one or more of the hydrogen atoms on the nitrogen is replaced by an alkyl group as defined above.

“Haloalkyl” means a halo-alkyl-group in which alkyl is as previously defined. Preferred haloalkyls contain lower alkyl.

“Alkoxyalkyl” means an alkoxy-alkyl group in which alkyl is as previously defined. Preferred alkoxyalkyls contain lower alkyl.

“Alkylsilyl” means an alkyl-Si-group in which alkyl is as previously defined and the point of attachment to the parent moiety is on Si. Preferred alkylsilyls contain lower alkyl.

Also included in the scope of this invention are oxidized forms of the heteroatoms (e.g., nitrogen and sulfur) that are present in the compounds of this invention. Such oxidized forms include N(O) [N⁺—O], S(O) and S(O)₂.

The term “isolated” or “in isolated form” for a compound refers to the physical state of said compound after being isolated from a synthetic process or natural source or combination thereof. The term “purified” or “in purified form” for a compound refers to the physical state of said compound after being obtained from a purification process or processes described herein or well known to the skilled artisan, in sufficient purity to be characterizable by standard analytical techniques described herein or well known to the skilled artisan.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

As used herein, the term “composition” is intended to encompass a product comprising the specified ingredients in the specified amounts, as well as any product which results, directly or indirectly, from combination of the specified ingredients in the specified amounts.

Isomers of the compounds of Formula I (where they exist), including enantiomers, stereoisomers, rotamers, diastereomers, tautomers and racemates are also contemplated as being part of this invention. The invention includes d and I isomers in both pure form and in admixture, including racemic mixtures. Isomers can be prepared using conventional techniques, either by reacting optically pure or optically enriched starting materials or by separating isomers of a compound of the Formula I. Isomers may also include geometric isomers, e.g., when a double bond is present. Polymorphous forms of the compounds of Formula I, whether crystalline or amorphous, also are contemplated as being part of this invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a ¹³C— or ¹⁴C-enriched carbon are also within the scope of this invention.

It will be apparent to one skilled in the art that certain compounds of this invention may exist in alternative tautomeric forms. All such tautomeric forms of the present compounds are within the scope of the invention. Unless otherwise indicated, the representation of either tautomer is meant to include the other. For example, both isomers (1) and (2) are contemplated: 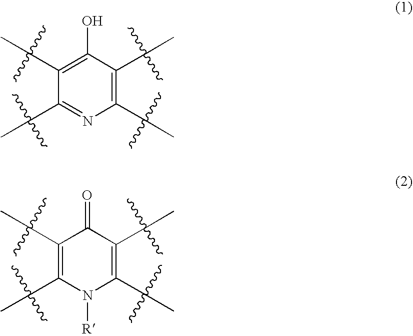
wherein R′ is H or C₁₋₆ unsubstituted alkyl.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of formula I or a salt, ester and/or solvate thereof (e.g., a prodrug on being brought to the physiological pH or through enzyme action is converted to the desired drug form). A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may also exist as, or optionally converted to, a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving a compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of a compound or a composition of the present invention effective in inhibiting mitotic kinesins, in particular KSP kinesin activity, and thus producing the desired therapeutic, ameliorative, inhibitory or preventative effect in a suitable subject.

The compounds of formula I form salts which are also within the scope of this invention. Reference to a compound of formula I herein is understood to include reference to salts, esters and solvates thereof, unless otherwise indicated. The term “salt(s)”, as employed herein, denotes acidic salts formed with inorganic and/or organic acids, as well as basic salts formed with inorganic and/or organic bases. In addition, when a compound of formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the formula I may be formed, for example, by reacting a compound of formula I with an amount of acid or base, such as an equivalent amount, in a medium such as one in which the salt precipitates or in an aqueous medium followed by lyophilization. Acids (and bases) which are generally considered suitable for the formation of pharmaceutically useful salts from basic (or acidic) pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Int'l. Union of Pure and Applied Chemistry, pp. 330-331. These disclosures are incorporated herein by reference thereto.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like.

Exemplary basic salts include ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl) ethylenediamine), N-methyl-D-glucamines, N-methyl-D-glucamides, t-butyl amines, piperazine, phenylcyclohexylamine, choline, tromethamine, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid salts and base salts are intended to be pharmaceutically acceptable salts within the scope of the invention. All acid and base salts, as well as esters and solvates, are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

In such esters, unless otherwise specified, any alkyl moiety present preferably contains from 1 to 18 carbon atoms, particularly from 1 to 6 carbon atoms, more particularly from 1 to 4 carbon atoms. Any cycloalkyl moiety present in such esters preferably contains from 3 to 6 carbon atoms. Any aryl moiety present in such esters preferably comprises a phenyl group.

Generally, the compounds of Formula I can be prepared by a variety of methods well known to those skilled in the art, for example, by the methods as outlined in Scheme 1 below and in the examples disclosed herein: 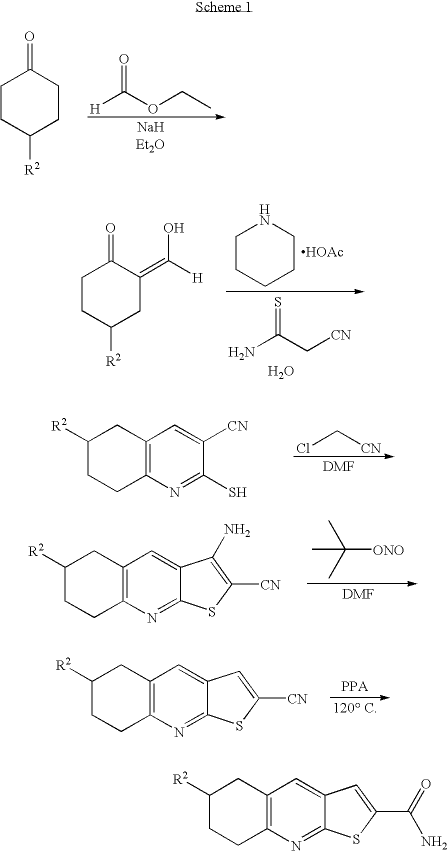
wherein R² is as defined above.

The compounds of the invention can be useful in a variety of applications involving alteration of mitosis. As will be appreciated by those skilled in the art, mitosis may be altered in a variety of ways; that is, one can affect mitosis either by increasing or decreasing the activity of a component in the mitotic pathway. Mitosis may be affected (e.g., disrupted) by disturbing equilibrium, either by inhibiting or activating certain components. Similar approaches may be used to alter meiosis.

In a particular embodiment, the compounds of the invention can be used to inhibit mitotic spindle formation, thus causing prolonged cell cycle arrest in mitosis. By “inhibit” in this context is meant decreasing or interfering with mitotic spindle formation or causing mitotic spindle dysfunction. By “mitotic spindle formation” herein is meant organization of microtubules into bipolar structures by mitotic kinesins. By “mitotic spindle dysfunction” herein is meant mitotic arrest and monopolar spindle formation.

The compounds of the invention can be useful for binding to, and/or inhibiting the activity of, a mitotic kinesin, KSP. In one embodiment, the KSP is human KSP, although the compounds may be used to bind to or inhibit the activity of KSP kinesins from other organisms. In this context, “inhibit” means either increasing or decreasing spindle pole separation, causing malformation, i.e., splaying, of mitotic spindle poles, or otherwise causing morphological perturbation of the mitotic spindle. Also included within the definition of KSP for these purposes are variants and/or fragments of KSP (see U.S. Pat. No. 6,437,115). In addition, the present compounds are also useful for binding to or modulating other mitotic kinesins.

The compounds of the invention can be used to treat cellular proliferation diseases. Such disease states which can be treated by the compounds, compositions and methods provided herein include, but are not limited to, cancer (further discussed below), hyperplasia, cardiac hypertrophy, autoimmune diseases, fungal disorders, arthritis, graft rejection, inflammatory bowel disease, immune disorders, inflammation, restenosis, cellular proliferation induced after medical procedures, including, but not limited to, surgery, angioplasty, and the like. Treatment includes inhibiting cellular proliferation. It is appreciated that in some cases the cells may not be in a hyper- or hypoproliferation state (abnormal state) and still require treatment. For example, during wound healing, the cells may be proliferating “normally”, but proliferation enhancement may be desired. Thus, in one embodiment, the invention herein includes application to cells or subjects afflicted or subject to impending affliction with any one of these disorders or states.

The compounds, compositions and methods provided herein are particularly useful for the treatment of cancer including solid tumors such as skin, breast, brain, colon, gall bladder, thyroid, cervical carcinomas, testicular carcinomas, etc. More particularly, cancers that may be treated by the compounds, compositions and methods of the invention include, but are not limited to:

Cardiac: sarcoma (angiosarcoma, fibrosarcoma, rhabdomyosarcoma, liposarcoma), myxoma, rhabdomyoma, fibroma, lipoma and teratoma;

Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;

Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);

Genitourinarv tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);

Liver: hepatoma (hepatocellular carcinoma), cholangiocarcinoma, hepatoblastoma, angiosarcoma, hepatocellular adenoma, hemangioma;

Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;

Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma);

Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);

Hematologic: blood (myeloid leukemia (acute and chronic), acute lymphoblastic leukemia, acute and chronic lymphocytic leukemia, myeloproliferative diseases, multiple myeloma, myelodysplastic syndrome), Hodgkin's disease, non-Hodgkin's lymphoma (malignant lymphoma), B-cell lymphoma, T-cell lymphoma, hairy cell lymphoma, Burkett's lymphoma, promyelocytic leukemia;

Skin: malignant melanoma, basal cell carcinoma, squamous cell carcinoma, Karposi's sarcoma, moles dysplastic nevi, lipoma, angioma, dermatofibroma, keloids, psoriasis;

Adrenal glands: neuroblastoma; and

Other tumors: including xenoderoma pigmentosum, keratoctanthoma and thyroid follicular cancer.

As used herein, treatment of cancer includes treatment of cancerous cells, including cells afflicted by any one of the above-identified conditions.

The compounds of the present invention may also be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

The compounds of the present invention may also be useful in inhibiting tumor angiogenesis and metastasis.

The compounds of the present invention may also be useful as antifungal agents, by modulating the activity of the fungal members of the bimC kinesin subgroup, as is described in U.S. Pat. No. 6,284,480.

The present compounds are also useful in combination with one or more other known therapeutic agents and anti-cancer agents. Combinations of the present compounds with other anti-cancer or chemotherapeutic agents are within the scope of the invention. Examples of such agents can be found in Cancer Principles and Practice of Oncology by V. T. Devita and S. Hellman (editors), 6th edition (Feb. 15, 2001), Lippincoft Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The present compounds are also useful when co-administered with radiation therapy.

The phrase “estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-I-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-ydrazone, aid SH646.

The phrase “androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

The phrase “retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, a difluoromethylornithine, ILX23-7553, trans-N-(4′-hydroxyphenyl) retinamide, and N-4-carboxyphenyl retinamide.

The phrase “cytotoxic/cytostatic agents” refer to compounds which cause cell death or inhibit cell proliferation primarily by interfering directly with the cell's functioning or inhibit or interfere with cell mycosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites; biological response modifiers; hormonal/anti-hormonal therapeutic agents, haematopoietic growth factors, monoclonal antibody targeted therapeutic agents, monoclonal antibody therapeutics, topoisomerase inhibitors, proteasome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide (TEMODAR™ from Schering-Plough Corporation, Kenilworth, N.J.), cyclophosphamide, heptaplatin, estramustine, improsulfan tosilate, trofosfamide, nimustine, dibrospidium chloride, pumitepa, lobaplatin, satraplatin, profiromycin, cisplatin, doxorubicin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum(II)] tetrachloride, diarizidinyispermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deansino-3′-morpholino-13-deoxo-10-hydroxycarminomycin, annamycin, galarubicin, elinafide, MEN10755, 4-demethoxy-3-deamino-3-aziridinyl-4-methylsulphonyl-daunombicin (see WO 00/50032), methoxtrexate, gemcitabine.

An example of a hypoxia activatable compound is tirapazamine.

Examples of proteasome inhibitors include, but are not limited to, lactacystin and bortezomib.

Examples of microtubule inhibitors/microtubule-stabilising agents include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl) benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H, 12H-benzo[de]pyrano[3′,4′:b,7]-indolizino[1,2b]quinoline-10,13(9H,15H)dione, lurtotecan, 7-[2-(N-isopropylamino) ethyl]-(20S)camptothecin, BNP1350, BNPI1100, BN80915, BN80942, etoposide phosphate, teniposide, sobuzoxane, 2′-d imethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydroxy-3,5-dimethoxyphenyl]-5,5a,6,8,8a ,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino] benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2-(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, dimesna, and camptostar.

Other useful anti-cancer agents that can be used in combination with the present compounds include thymidilate synthase inhibitors, such as 5-fluorouracil.

In one embodiment, inhibitors of mitotic kinesins include, but are not limited to, inhibitors of KSP, inhibitors of MKLP1, inhibitors of CENP-E, inhibitors of MCAK, inhibitors of Kif14, inhibitors of Mphosph1 and inhibitors of Rab6-KIFL.

The phrase “inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

The phrase “antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, neizarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

Examples of monoclonal antibody therapeutics useful for treating cancer include Erbitux (Cetuximab).

The phrase “HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin(ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefore the use of such salts, esters, open acid and lactone forms is included in the scope of this invention.

The phrase “prenyl-protein transferase inhibitor” refers to a compound which inhibits any one or any combination of the prenyl-protein transferase enzymes, including famesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. Nos. 5,420,245, 5,523,430, 5,532,359, 5,510,510, 5,589,485, 5,602,098, European Patent Publ. 0 618 221, European Patent Publ. 0 675 112, European Patent Publ. 0 604181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO, 97/30053, WO 97/44350, WO 98/02436, and U.S. Pat. No. 5,532,359. For an example of the role of a prenyl-protein transferase inhibitor on angiogenesis see European of Cancer, Vol. 35, No. 9, pp. 1394-1401(1999).

Examples of farnesyl protein transferase inhibitors include SARASAR™ (4-[2-[4-[(11R)-3,10-dibromo-8-chloro-6,11-dihydro-5H-benzo[5,6]cyclohepta[1,2-b]pyridin-11-yl-]-1-piperidinyl]-2-oxoehtyl]-1-piperidinecarboxamide from Schering-Plough Corporation, Kenilworth, N.J.), tipifarnib (Zarnestra® or R115777 from Janssen Pharmaceuticals), L778,123 (a farnesyl protein transferase inhibitor from Merck & Company, Whitehouse Station, N.J.), BMS 214662 (a farnesyl protein transferase inhibitor from Bristol-Myers Squibb Pharmaceuticals, Princeton, N.J.).

The phrase “angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α (for example Intron and Peg-Intron), interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin. Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). Examples of TAFIa inhibitors have been described in PCT Publication WO 03/013,526.

The phrase “agents that interfere with cell cycle checkpoints” refers to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

The phrase “inhibitors of cell proliferation and survival signaling pathway” refers to agents that inhibit cell surface receptors and signal transduction cascades downstream of those surface receptors. Such agents include inhibitors of EGFR (for example gefitinib and erlotinib), antibodies to EGFR (for example C225), inhibitors of ERB-2 (for example trastuzumab), inhibitors of IGFR, inhibitors of cytokine receptors, inhibitors of MET, inhibitors of PI3K (for example LY294002), serine/threonine kinases (including but not limited to inhibitors of Akt such as described in WO 02/083064, WO 02/083139, WO 02/083140 and WO 02/083138), inhibitors of Raf kinase (for example BAY-43-9006), inhibitors of MEEK (for example CI-1040 and PD-098059), inhibitors of mTOR (for example Wyeth CCI-779), and inhibitors of C-abl kinase (for example GLEEVEC™, Novartis Pharmaceuticals). Such agents include small molecule inhibitor compounds and antibody antagonists.

The phrase “apoptosis inducing agents” includes activators of TNF receptor family members (including the TRAIL receptors).

The invention also encompasses combinations with one or more NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC50 for COX-2 over IC50 for COX-1 evaluated by cell or microsomal assays. Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5 pyridinyl)pyridine; or a pharmaceutically acceptable salt thereof.

Compounds that have been described as specific inhibitors of COX-2 and are therefore useful in the present invention include, but are not limited to, parecoxib, CELEBREX® and BEXTRA® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some examples of tyrosine kinase inhibitors include N-(trifluoromethylphenyl)-5-methylisoxazol-4-carboxamide, 3-[(2,4-dimethylpyrrol-5-yl)methylidenyl)indolin-2-one,17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′- kl]pyrrolo[3,4-i][1,6]benzodiazocin-1-one, SH268, genistein, ST1571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, ST1571A, N-4-chlorophenyl-4-(4-pyridylmethyl)-1-phthalazinamine, and EMD121974.

Combinations with compounds other than anti-cancer compounds are also encompassed in the instant methods. For example, combinations of the present compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999;274:9116-9121; Invest. Ophthalmol Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid, and 2(R)-7-(3-(2-chloro-4-(4-fluorophenoxy) phenoxy)propoxy)-2-ethylchromane-2-carboxylic acid.

In one embodiment, useful anti-cancer (also known as anti-neoplastic) agents that can be used in combination with the present compounds include, but are not limited, to Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin (ELOXATIN™ from Sanofi-Synthelabo Pharmaeuticals, France), Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin (adriamycin), cyclophosphamide (cytoxan), gemcitabine, interferons, pegylated interferons, Erbitux and mixtures thereof.

Another embodiment of the present invention is the use of the present compounds in combination with gene therapy for the treatment of cancer. For an overview of genetic strategies to treating cancer, see Hall et al (Am J Hum Genet 61:785-789,1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998;5(8):1105-13), and interferon gamma (J Immunol 2000;1 64:217-222).

The present compounds can also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

The present compounds can also be employed in conjunction with one or more anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with one or more other anti-emetic agents, especially neurokinin-1 receptor antagonists, 5HT3 receptor, antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or those as described in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In one embodiment, an anti-emesis agent selected from a neurokinin-1 receptor antagonist, a 5HT3 receptor antagonist and a corticosteroid is administered as an adjuvant for the treatment or prevention of emesis that may result upon administration of the present compounds.

Examples of neurokinin-1 receptor antagonists that can be used in conjunction with the present compounds are described in U.S. Pat. Nos. 5,162,339, 5,232,929, 5,242,930, 5,373,003, 5,387,595, 5,459,270, 5,494,926, 5,496,833, 5,637,699, and 5,719,147, content of which are incorporated herein by reference. In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the present invention may also be administered with one or more immunologic-enhancing drugs, such as levamisole, isoprinosine and Zadaxin.

Thus, the present invention encompasses the use of the present compounds (for example, for treating or preventing cellular proliferative diseases) in combination with a second compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of inherent multidrug resistance, an anti-emetic agent, an immunologic-enhancing drug, an inhibitor of cell proliferation and survival signaling, an agent that interfers with a cell cycle checkpoint, and an apoptosis inducing agent.

In one embodiment, the present invention empassesses the composition and use of the present compounds in combination with a second compound selected from: a cytostatic agent, a cytotoxic agent, taxanes, a topoisomerase II inhibitor, a topoisomerase I inhibitor, a tubulin interacting agent, hormonal agent, a thymidilate synthase inhibitors, anti-metabolites, an alkylating agent, a farnesyl protein transferase inhibitor, a signal transduction inhibitor, an EGFR kinase inhibitor, an antibody to EGFR, a C-abl kinase inhibitor, hormonal therapy combinations, and aromatase combinations.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In one embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MW (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-(O-chloroacetylcarbonyl)-fumagillol, thalidomide, angiostatin, troponin-1, or an antibody to VEGF. In an embodiment, the estrogen receptor modulator is tamoxifen or raloxifene.

Also included in the present invention is a method of treating cancer comprising administering a therapeutically effective amount of at least one compound of Formula I in combination with radiation therapy and at least one compound selected from: an estrogen receptor modulator, an androgen receptor modulator, retinoid receptor modulator, a cytotoxictcytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of inherent multidrug resistance, an anti-emetic agent, an immunologic-enhancing drag, an inhibitor of cell proliferation and survival signaling, an agent that interfers with a cell cycle checkpoint, and an apoptosis inducing agent.

Yet another embodiment of the invention is a method of treating cancer comprising administering a therapeutically effective amount of at least one compound of Formula I in combination with paclitaxel or trastuzumab.

The present invention also includes a pharmaceutical composition useful for treating or preventing cellular proliferation diseases (such as cancer, hyperplasia, cardiac hypertrophy, autoimmune diseases, fungal disorders, arthritis, graft rejection, inflammatory bowel disease, immune disorders, inflammation, restenosis and cellular proliferation induced after medical procedures) that comprises a therapeutically effective amount of at least one compound of Formula I and at least one compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of cell proliferation and survival signaling, an agent that interfers with a cell cycle checkpoint, and an apoptosis inducing agent.

Another aspect of this invention relates to a method of selectively inhibiting KSP kinesin activity in a subject (such as a cell, animal or human) in need thereof, comprising contacting said subject with at least one compound of Formula I or a pharmaceutically acceptable salt or ester thereof.

Preferred KSP kinesin inhibitors are those which can specifically inhibit KSP kinesin activity at low concentrations, for example, those that cause a level of inhibition of 50% or greater at a concentration of 50 μM or less, more preferably 100 nM or less, most preferably 50 nM or less.

Another aspect of this invention relates to a method of treating or preventing a disease or condition associated with KSP in a subject (e.g., human) in need thereof comprising administering a therapeutically effective amount of at least one compound of Formula I or a pharmaceutically acceptable salt or ester thereof to said subject.

A preferred dosage is about 0.001 to 500 mg/kg of body weight/day of a compound of Formula I or a pharmaceutically acceptable salt or ester thereof. An especially preferred dosage is about 0.01 to 25 mg/kg of body weight/day of a compound of Formula I or a pharmaceutically acceptable salt or ester thereof.

The phrases “effective amount” and “therapeutically effective amount” mean that amount of a compound of Formula I, and other pharmacological or therapeutic agents described herein, that will elicit a biological or medical response of a tissue, a system, or a subject (e.g., animal or human) that is being sought by the administrator (such as a researcher, doctor or veterinarian) which includes alleviation of the symptoms of the condition or disease being treated and the prevention, slowing or halting of progression of one or more cellular proliferation diseases. The formulations or compositions, combinations and treatments of the present invention can be administered by any suitable means which produce contact of these compounds with the site of action in the body of, for example, a mammal or human.

For administration of pharmaceutically acceptable salts of the above compounds, the weights indicated above refer to the weight of the acid equivalent or the base equivalent of the therapeutic compound derived from the salt.

As described above, this invention includes combinations comprising an amount of at least one compound of Formula I or a pharmaceutically acceptable salt or ester thereof, and an amount of one or more additional therapeutic agents listed above (administered together or sequentially) wherein the amounts of the compounds/treatments result in desired therapeutic effect.

If formulated as a fixed dose, such combination products employ the compounds of this invention within the dosage range described herein and the other pharmaceutically active agent or treatment within its dosage range. Compounds of Formula I may also be administered sequentially with known therapeutic agents when a combination formulation is inappropriate. The invention is not limited in the sequence of administration; compounds of Formula I may be administered either prior to or after administration of the known therapeutic agent. Such techniques are within the skills of persons skilled in the art as well as attending physicians.

The pharmacological properties of the compounds of this invention may be confirmed by a number of pharmacological assays. The inhibitory activity of the present compounds towards KSP may be assayed by methods known in the art, for example, by using the methods as described in the examples.

While it is possible for the active ingredient to be administered alone, it is preferable to present it as a pharmaceutical composition. The compositions of the present invention comprise at least one active ingredient, as defined above, together with one or more acceptable carriers, adjuvants or vehicles thereof and optionally other therapeutic agents. Each carrier, adjuvant or vehicle must be acceptable in the sense of being compatible with the other ingredients of the composition and not injurious to the mammal in need of treatment.

Accordingly, this invention also relates to pharmaceutical compositions comprising at least one compound of Formula I, or a pharmaceutically acceptable salt or ester thereof and at least one pharmaceutically acceptable carrier, adjuvant or vehicle.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

The term pharmaceutical composition is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a subject by administering a pharmaceutical composition of the present invention is also intended to encompass the administration of the afore-said bulk composition and individual dosage units.

Additionally, the compositions of the present invention may be formulated in sustained release form to provide the rate controlled release of any one or more of the components or active ingredients to optimize the therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts or esters thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula I or a pharmaceutically acceptable salt or ester thereof and at least one pharmaceutically acceptable carrier, adjuvant or vehicle.

Yet another aspect of this invention is a kit comprising an amount of at least one compound of Formula I or a pharmaceutically acceptable salt or ester thereof and an amount of at least one additional therapeutic agent listed above, wherein the amounts of the two or more ingredients result in desired therapeutic effect.

The invention disclosed herein is exemplified by the following preparations and examples which should not be construed to limit the scope of the disclosure. Alternative mechanistic pathways and analogous structures will be apparent to those skilled in the art.

The following solvents and reagents may be referred to by their abbreviations in parenthesis:
Thin layer chromatography: TLC
dichloromethane: CH₂Cl₂
ethyl acetate: AcOEt or EtOAc
methanol: MeOH
trifluoroacetate: TFA
triethylamine: Et₃N or TEA
butoxycarbonyl: n-Boc or Boc
nuclear magnetic resonance spectroscopy: NMR
liquid chromatography mass spectrometry: LCMS
high resolution mass spectrometry: HRMS
milliliters: mL
millimoles: mmol
microliters: μl
grams: g
milligrams: mg
room temperature or rt (ambient): about 25° C.
dimethoxyethane: DME
N, N-Dimethylformamide: DMF
4-Methylmorpholine: NMM
O-(7-Azabenzotriazol-1-yl)-N, N, N′, N′-tetramethyluronium PF₆: HATU

### EXAMPLES

Illustrating the invention are the following examples which, however, are not to be considered as limiting the invention to their details. Unless otherwise indicated, all parts and percentages in the following examples, as well as throughout the specification, are by weight.

### Example 1

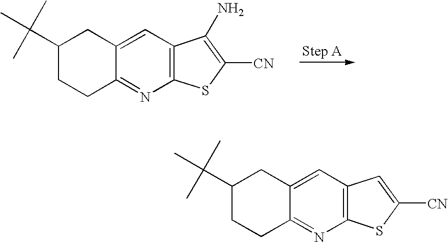 Step A:

6-tert-Butyl-5,6,7,8-tetrahydrothieno[2,3-b]quinoline-2-carbonitrile: To a solution of 90% t-butylnitrite (526 mg, 4.60 mmol) in 6 mL of DMF stirred at 65° C., was added a solution of 3-amino-6-tert-butyl-5,6,7,8-tetrahydrothieno[2,3-b]quinoline-2-carbonitrile (820 mg, 2.87 mmol) in 6 mL of DMF dropwise. The reaction was stirred at 65° C. for 30 min. Upon cooling to room temperature, it was added into 100 mL of H₂O. This was extracted by 100 mL of EtOAc. The organic phase was dried over anhydrous Na₂SO₄ and then concentrated. The residue was purified by flash chromatography eluting with 15% EtOAc/hexanes to give 500 mg (64%) of 6-tert-butyl-5,6,7,8-tetrahydrothieno[2,3-b]quinoline-2-carbonitrile. LCMS: MH⁺=271; mp (° C.)=133-135. 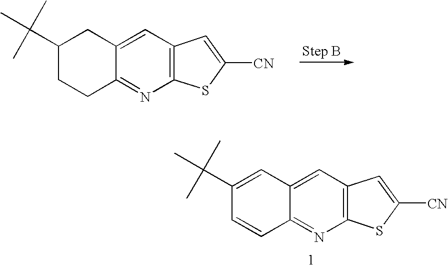
Step B:

6-tert-Bulyl-thieno[2,3-b]quinoline-2-carbonitrile (1): To a solution of 6-tert-butyl-5,6,7,8-tetrahydrothieno[2,3-b]quinoline-2-carbonitrile (2.0 g, 7.4 mmol) in 50 mL of toluene, was added 2,3-dichloro-5,6-dicyano-1,4-benzoquinone (4.20 g, 18.5 mmol). The reaction mixture was refluxed under nitrogen for 17 h. Upon cooling to room temperature, it was diluted with 50 mL of CH₂Cl₂. The resulting mixture was filtered through Celite. The mother liquor was concentrated under vacuum. To the residue was added 100 mL of CH₂Cl₂. The resulting mixture was washed with 50 mL of 1 N aqueous NaOH, and 50 mL of H₂O. The organic phase was concentrated under vacuum. The residue was further purified by flash chromatography eluting with CH₂Cl₂ to give 950 mg (48%) of 6-tert-butyl-thieno[2,3-b]quinoline-2-carbonitrile. LCMS: MH⁺=267; mp (° C.)=150-152.

### Example 2

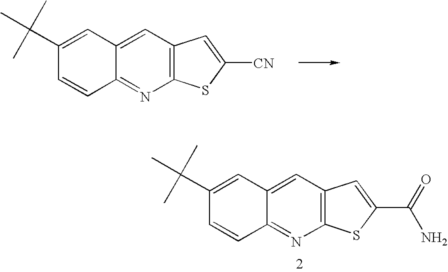

6-tert-Butyl-thieno[2,3-b]quinoline-2-carboxylic acid amide(2): A mixture of 6-tert-butyl-thieno[2,3-b]quinoline-2-carbonitrile (46 mg, 0.172 mmol) in 3 g of polyphosphoric acid was stirred at 120° C. for 5 h. After it was cooled to room temperature, 30 mL of ice H₂O was added. It was stirred at room temperature for 15 min. The mixture was neutralized by 2 N aqueous NaOH. The solid was collected by filtration. It was then dissolved in 20 mL of 5% MeOH/CH₂Cl₂, washed with 15 mL of 2 N aqueous Na₂CO₃ and then concentrated. The residue was further purified by flash chromatography eluting with 10% MeOH/CH₂Cl₂ to give 46 mg (94%) of 6-tert-butyl-thieno[2,3-b]quinoline-2-carboxylic acid amide. LCMS: MH⁺=285; mp (° C.)=239-264 (dec.).

### Example 3

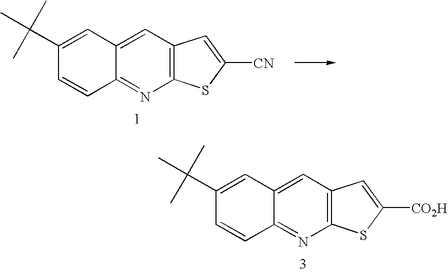

6-tert-Butyl-thieno[2,3-b]quinoline-2-carboxylic acid (3): A mixture of 6-tert-butyl-thieno[2,3-b]quinoline-2-carbonitrile (250 mg, 0.94 mmol) in 5 mL of 85% phosphoric acid was stirred at 160° C. for 4.5 h. After it was cooled to room temperature, 100 mL of ice H₂O was added. The pH of which was adjusted to 5 by 2 N aqueous NaOH. The solid was collected by filtration. It was washed with H₂O, then with CH₂Cl₂/hexanes (1:1) and dried under vacuum to give 242 mg (90%) of 6-tert-butyl-thieno[2,3-b]quinoline-2-carboxylic acid. LCMS: MH⁺=286; mp (° C.)=289-292 (dec.).

### Example 4

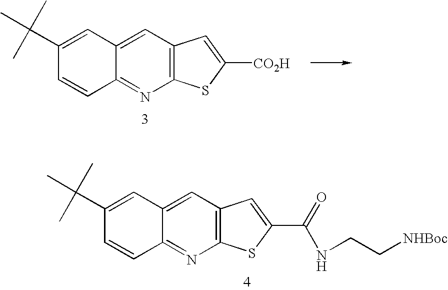

{2-[(6-tert-Butyl-thieno[2,3-b]quinoline-2-carbonyl)amino]ethyl}carbamic acid tert-butyl ester (4): To a solution of 6-tert-butyl-thieno[2,3-b]quinoline-2-carboxylic acid (56 mg, 0.20 mmol) in 2 mL of thionyl chloride/CH₂Cl₂ (1:1), was added one drop of DMF. The reaction was stirred at 40° C. for 2 h. The solvent was removed under vacuum. To the residue was added 2 mL of toluene. The resulting mixture was concentrated under vacuum to remove any residual thionyl chloride. To the residue was added 2 mL of CH₂Cl₂ followed by a solution of (2-aminoethyl)carbamic acid tert-butyl ester (38 mg, 0.24 mmol) and triethylamine (95 mg, 0.94 mmol) in 2 mL of CH₂Cl₂. The reaction was stirred at room temperature for 1 h. It was diluted with 20 mL of CH₂Cl₂, and washed with 1 N aqueous HCl (10 mL) and 2 N aqueous NaHCO₃ (10 mL). The organic phase was dried over anhydrous Na₂SO₄ and then concentrated under vacuum. The residue was recrystallized from CH₂Cl₂/hexanes to give 65 mg (78%) of {2-[(6-tert-butyl-thieno[2,3-b]quinoline-2-carbonyl)amino]ethyl} carbamic acid tert-butyl ester. LCMS: MH⁺=428; mp (° C.)=212-217 (dec.).

### Example 5

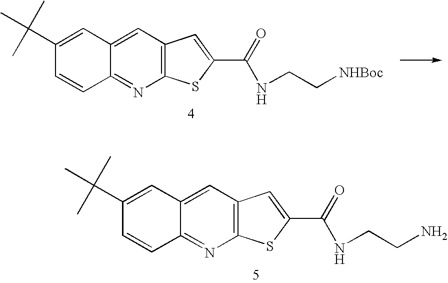

6-tert-Butyl-thieno[2,3-b]quinoline-2-carboxylic acid (2-aminoethyl)amide (5): To a solution of {2-[(6-tert-butyl-thieno[2,3-b]quinoline-2-carbonyl)amino]ethyl}carbamic acid tert-butyl ester (47 mg, 0.11 mmol) in 3 mL of CH₂Cl₂, was added 1.5 mL of trifluoroacetic acid. The reaction was stirred at room temperature for 2 h. It was concentrated under vacuum. The residue was diluted with 20 mL of CH₂Cl₂, washed with 10 mL of saturated aqueous NaHCO₃ and dried over anhydrous Na₂SO₄. The solvent was removed under vacuum to give 35 mg (97%) of 6-tert-butyl-thieno[2,3-b]quinoline-2-carboxylic acid (2-aminoethyl)amide. LCMS: MH⁺=328; mp (° C.)=94-210 (dec.).

### Example 6

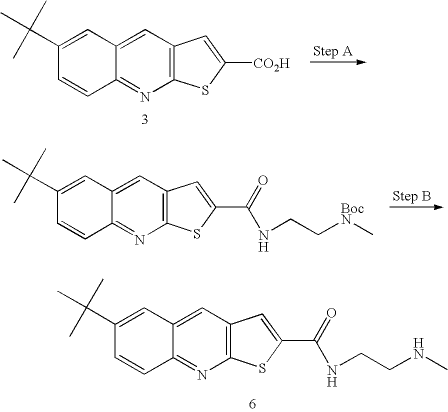 Step A

{2-[(6-tert-Butyl-thieno[2,3-b]quinoline-2-carbonyl)amino]ethyl}methyl carbamic acid tert-butyl ester: To a solution of 6-tert-butyl-thieno[2,3-b]quinoline-2-carboxylic acid (35 mg, 0.12 mmol) in 3 mL of thionyl chloride/CH₂Cl₂ (1:1), was added catalytic amount of DMF. The reaction was stirred at 40° C. for 2 h. The solvent was removed under vacuum. To the residue was added 2 mL of toluene. The resulting mixture was concentrated under vacuum to remove any residual thionyl chloride. To the residue was added 2 mL of CH₂Cl₂ followed by a solution of (2-aminoethyl)methylcarbamic acid tert-butyl ester (28 mg, 0.16 mmol), diisopropylethylamine (63.5 mg, 0.49 mmol) in 1 mL of CH₂Cl₂. The reaction was stirred at room temperature for 1 h. It was diluted with 20 mL of CH₂Cl₂, and washed with 1 N aqueous HCl (10 mL), saturated aqueous NaHCO₃ (10 mL). The organic phase was then concentrated under vacuum. The residue was further purified by flash chromatography eluting with 5% MeOH/CH₂Cl₂ to give 53 mg (98%) of {2-[(6-tert-butyl-thieno[2,3-b]quinoline-2-carbonyl)amino]ethyl}methylcarbamic acid tert-butyl ester.

Step B:

6-tert-Butyl-thieno[2,3-b]quinoline-2-carboxylicacid (2-methylaminoethyl) amide (6): To a solution of {2-[(6-tert-butyl-thieno[2,3-b]quinoline-2-carbonyl) amino]-ethyl}-methylcarbamic acid tert-butyl ester (52 mg, 0.12 mmol) in 1.5 mL of CH₂Cl₂, was added 1.0 mL of trifluoroacetic acid. The reaction was stirred at room temperature for 3 h. It was concentrated under vacuum. The residue was diluted with 15 mL of CH₂Cl₂, washed with 10 mL of saturated aqueous NaHCO₃. The organic layer was concentrated under vacuum. The residue was recrystallized from CH₂Cl₂/hexanes to give 26 mg of 6-tert-butyl-thieno[2,3-b]quinoline-2-carboxylic acid (2-methylaminoethyl)amide. LCMS: MH⁺=342; mp (° C.)=153-155.

### Example 7

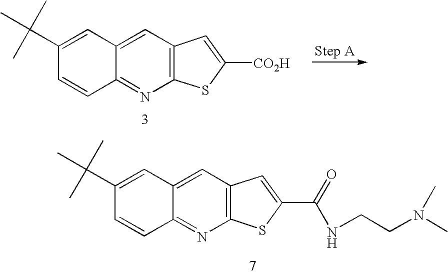

6-tert-Butyl-thieno[2,3-b]quinoline-2-carboxylic acid (2-dimethylaminoethyl)-amide (7): To a solution of 6-tert-butyl-thieno[2,3-b]quinoline-2-carboxylic acid (25 mg, 0.088 mmol) in 2 mL of thionyl chloride/CH₂Cl₂ (1:1), was added catalytic amount of DMF. The reaction was stirred at 40° C. for 1.5 h. The solvent was removed under vacuum. To the residue was added 2 mL of toluene. The resulting mixture was concentrated under vacuum to remove any residual thionyl chloride. To the residue was added 2 mL of CH₂Cl₂ followed by a solution of N,N-dimethylethylenediamine (11.5 mg, 0.13 mmol), diisopropylethylamine (46 mg, 0.36 mmol) in 0.5 mL of CH₂Cl₂. The reaction was stirred at room temperature for 1 h. It was diluted with 20 mL of CH₂CI2, and washed with 1 N aqueous HCl (10 mL), 1 N aqueous NaOH (10 mL). The organic phase was then concentrated under vacuum. The residue was further purified by flash chromatography eluting with 25% MeOH/CH₂Cl₂ to give 29 mg (93%) of 6-tert-butyl-thieno[2,3-b]quinoline-2-carboxylic acid (2-dimethylaminoethyl)-amide. LCMS: MH⁺=356; mp (° C.)=113-118.

### Example 8

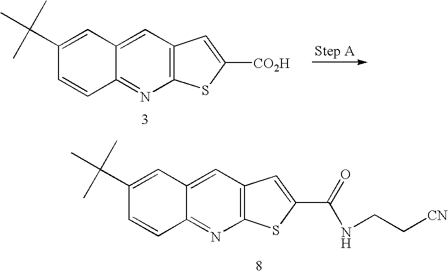

6-tert-Butyl-thieno[2,3-b]quinoline-2-carboxylic acid (2-cyanoethyl)amide (8): To a solution of 6-tert-butyl-thieno[2,3-b]quinoline-2-carboxylic acid (50 mg, 0.18 mmol) in 3 mL of thionyl chloride/CH₂Cl₂ (1:1), was added catalytic amount of DMF. The reaction was stirred at 40° C. for 2 h. The solvent was removed under vacuum. To the residue was added 2 mL of toluene. The resulting mixture was concentrated under vacuum to remove any residual thionyl chloride. To the residue was added 2 mL of CH₂Cl₂ followed by a solution of 3-aminopropionitrile (18.4 mg, 0.26 mmol), diisopropylethylamine (90 mg, 0.70 mmol) in 0.5 mL of CH₂Cl₂. The reaction was stirred at room temperature for 1 h. To the reaction mixture was added 1 mL of hexanes. The solid was collected by filtration to give 42 mg (71%) of 6-teft-butyl-thieno[2,3-b]quinoline-2-carboxylic acid (2-cyanoethyl)amide. LCMS: MH⁺=338; mp (° C.)=258-260 (dec.).

### Example 9

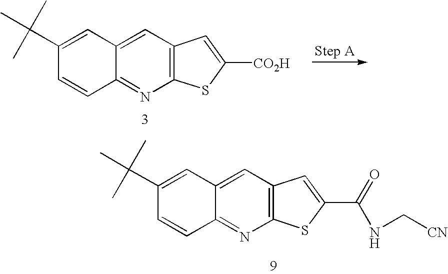

6-tert-Butyl-thieno[2,3-b]quinoline-2-carboxylicacid cyanomethylamide (9): To a mixture of 6-tert-butyl-thieno[2,3-b]quinoline-2-carboxylic acid (62 mg, 0.22 mmol) and aminoacetonitrile bisulfate (470 mg, 3.05 mmol), a solution of O(7-azabenzotriazol-1-yl)-N, N, N′, N′-tetramethyluronium hexafluorophosphate (525 mg, 1.38 mmol) in 4.5 mL of DMF was added. This was followed the addition of N-methyl morpholine (442 mg, 4.37 mmol). The reaction was stirred at room temperature overnight. It was diluted with 30 mL of water, extracted by 30 mL of 90% AcOEt/hexanes. The organic layer was dried over anhydrous Na₂SO₄ and then concentrated. The residue was further purified by flash chromatography eluting with 5% MeOH/CH₂Cl₂ to give 65 mg (92%) of 6-tert-butyl-thieno[2,3-b]quinoline-2-carboxylic acid cyanomethylamide. LCMS: MH⁺=324; mp (° C.)=224-225.

### Example 10

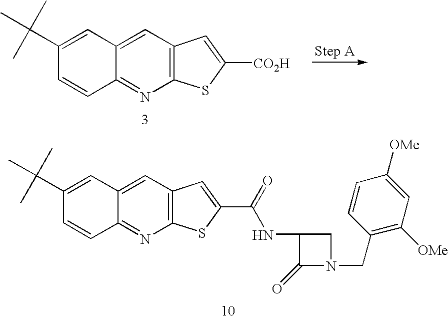

6-tert-Butyl-thieno[2,3-b]quinoline-2-carboxylic acid [1-(2,4-dimethoxybenzyl)-2-oxo-azetidin-3-yl]amide (10): To a solution of 6-tert-butyl-thieno[2,3-b]quinoline-2-carboxylic acid (40 mg, 0.14 mmol) in 2 mL of thionyl chloride/CH₂Cl₂ (1:1), was added catalytic amount of DMF. The reaction was stirred at 40° C. for 2 h. The solvent was removed under vacuum. To the residue was added 2 mL of toluene. The resulting mixture was concentrated under vacuum to remove any residual thionyl chloride. To the residue was added 2 mL of CH₂Cl₂ followed by a solution of 3-amino-1-(2,4-dimethoxybenzylyazetidin-2-one (42 mg, 0.18 mmol; for the preparation of this compound, see: Overman, L. E.; Osawa, T. J. Am. Chem. Soc. 1985, 107, 1698-701), diisopropylethylamine (72 mg, 0.56 mmol) in 0.5 mL of CH₂Cl₂. The reaction was stirred at room temperature for 1 h. It was diluted with 20 mL of CH₂Cl₂, and washed with 1 N aqueous HCl (20 mL), saturated aqueous NaHCO₃ (10 mL). The organic phase was then concentrated under vacuum. The residue was further purified by flash chromatography eluting with 4% MeOH/CH₂Cl₂ to give 65 mg (92%) of 6-tert-butyl-thieno[2,3-b]quinoline-2-carboxylic acid [1-(2,4-dimethoxybenzyl)-2-oxo-azetidin-3-yl]amide. LCMS: MH⁺=504; mp (° C.)=119-130 (dec.).

### Example 11

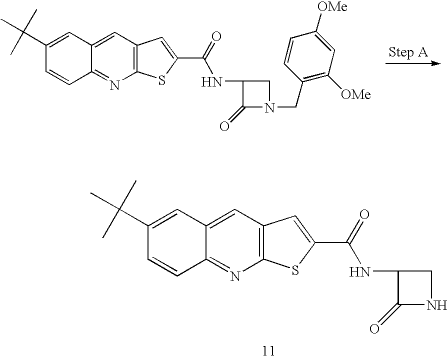

6-tert-Butyl-thieno[2,3-b]quinoline-2-carboxylic acid (2-oxo-azetidin-3-yl)amide: To a solution of 6-tert-butyl-thieno[2,3-b]quinoline-2-carboxylic acid [1-(2,4-dimethoxybenzyl)-2-oxo-azetidin-3-yl]amide (46 mg, 0.091 mmol) in 5 mL of acetonitrile/water (9:1), was added ceric ammonium nitrate (300 mg, 0.55 mmol). The reaction was stirred at room temperature for 15 min. It was diluted with 30 mL of CH₂Cl₂, and washed with water (15 mL), The aqueous layer was back extracted by 20 mL of CH₂Cl₂. The combined organic phase was then concentrated under vacuum. The residue was further purified by flash chromatography eluting with 6% MeOH/CH₂Cl₂ to give 21 mg (65%) of 6-tert-butyl-thieno[2,3-b]quinoline-2-carboxylic acid (2-oxo-azetidin-3-yl)amide. LCMS: MH⁺=354; mp (° C.)=169-185 (dec.).

### Example 12

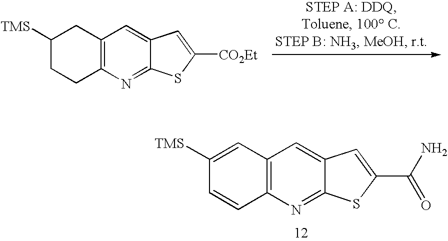

6-(TRIMETHYLSILYL)THIENO[2,3-b]QUINOLINE-2-CARBOXAMIDE (12): STEP A: To a solution of ethyl 5,6,7,8-tetrahydro-6-(trimethylsilyl)thieno[2,3-b]quinoline-2-carboxylate (330 mg, 0.99 mmol) in toluene (15ml) at r.t., DDQ (898 mg, 3.96 mmol) was added in small portions. The mixture was heated at 100° C. overnight. After being cooled to r.t., the solid was filtered through Celite. Solvents were removed in vacuum to give a red oil. Column purification [Hexanes—ethyl acetate, 9:1 (v/v)] gave ethyl 6-(trimethylsilyl)thieno[2,3-b]quinoline-2-carboxamide (160 mg,49%) as white solid. Electrospray LCMS [M+1]⁺=330.

STEP B: To a solution of ethyl 6-(trimethylsilyl)thieno[2,3-b]quinoline-2-carboxamide (160 mg, 0.49 mmol) in methanol (20 ml) at 0° C., ammonia was bubble through the solution for 30 min. The mixture was stirred in a sealed-tube overnight. Removal of solvents in vacuum gave a white solid. Column purification [Hexanes—ethyl acetate, 1:1 (v/v)] gave pure 6-(trimethylsilyl)thieno[2,3-b]quinoline-2-carboxamide (60 mg, 41%) as white solid. Electrospray LCMS [M+1]⁺=301.

### Example 13

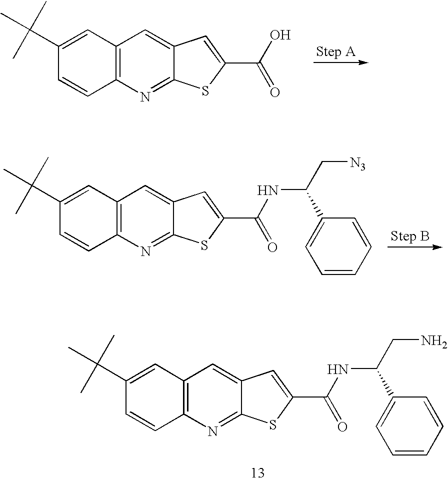 Step A:

6-tert-Butyl-thieno[2,3-b]quinoline-2-carboxylic acid (2-azido-1-(S)-phenyl-ethyl)-amide: To a solution of 6-tert-butyl-thieno[2,3-b]quinoline-2-carboxylic acid (250 mg, 0.86 mmol) in 6 mL of thionyl chloride/CH₂Cl₂ (1:1.5), was added catalytic amount of DMF (3 drops). The reaction was stirred at 40° C. for 2 h. The solvent was removed under vacuum. To the residue was added 2 mL of toluene. The resulting mixture was concentrated under vacuum to remove any residual thionyl chloride. To the residue was added 4 mL of CH₂Cl₂ followed by a solution of 2-azido-1-phenyl-ethylamine (140 mg, 0.86 mmol), diisopropylethylamine (223 mg, 1.74 mmol) in 4 mL of CH₂Cl₂. The reaction was stirred at room temperature for 1 h. It was diluted with 20 mL of CH₂Cl₂, and washed with 1 N aqueous HCl (20 mL), saturated aqueous NaHCO₃ (10 mL). The organic phase was then concentrated under vacuum. The residue was further purified by flash chromatography eluting with 10% EtOAc/CH₂Cl₂ to give 315 mg (84%) of 6-tert-butyl-thieno[2,3-b]quinoline-2-carboxylic acid (2-azido-1-(S)-phenyl-ethyl)-amide.

Step B:

6-tert-Butyl-thieno[2.3-b]quinoline-2-carboxylic acid (2-amino-1-(S)-phenyl-ethyl)-amide (13): To a solution of 6-tert-butyl-thieno[2,3-b]quinoline-2-carboxylic acid (2-azido-1-(S)-phenyl-ethyl)-amide (315 mg, 0.73 mmol) in 7 mL of MeOH was added 10% Pd/C (280 mg). The mixture was stirred under 1 atm of H₂ (gas) for 1.5 h. The mixture was filtered through a pad of Celite and the filtrate was concentrated in vacuo. The residue was purified via preparative TLC eluting with 10% MeOH/CH₂Cl₂ to give 113 mg (38%) of the hydrochloride salt of 6-tert-butyl-thieno[2,3-b]quinoline-2-carboxylic acid (2-amino-1-(S)-phenyl-ethyl)-amide. LCMS: MH⁺=404; mp (° C.)=203-208.

### Example 14

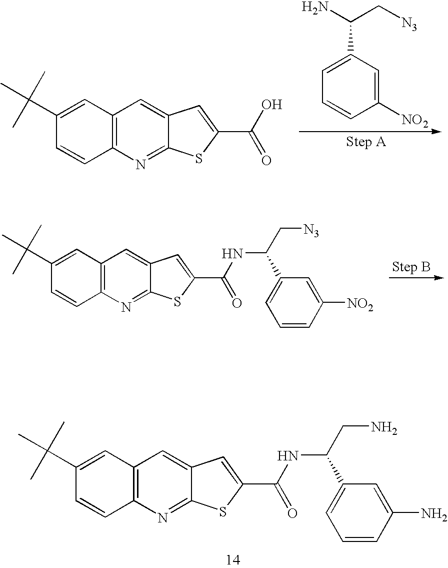 Step A:

6-tert-Butyl-thieno[2,3-b]quinoline-2-carboxylic acid [2(S)-azido-1-(3-nitro-phenyl)-ethyl]-amide: Following the same procedure set forth in example 12, step A, only substituting the amine with (1S)-2-azido-1-(3-nitrophenyl)-ethylamine gave 6-tert-butyl-thieno[2,3-b]quinoline-2-carboxylic acid [2(S)-azido-1-(3-nitro-phenyl)-ethyl]-amide. LCMS: MH⁺=475; mp (° C.)=79-87.

Step B:

6-tert-Butyl-thieno[2,3-biquinoline-2-carboxylic acid [2(S)-amino-1-(3-amino-phenyl)-ethyl]-amide (14): Following the same procedure set forth in example 12, step B, only substituting the with 6-tert-butyl-thieno[2,3-b]quinoline-2-carboxylic acid [2(S)-azido-1-(3-nitro-phenyl)-ethyl]-amide gave 6-tert-butyl-thieno[2,3-b]quinoline-2-carboxylic acid [2(S)-amino-1-(3-amino-phenyl)-ethyl]-amide. LCMS: MH⁺=419; mp (° C.)=185-201 (dec.).

### Example 15

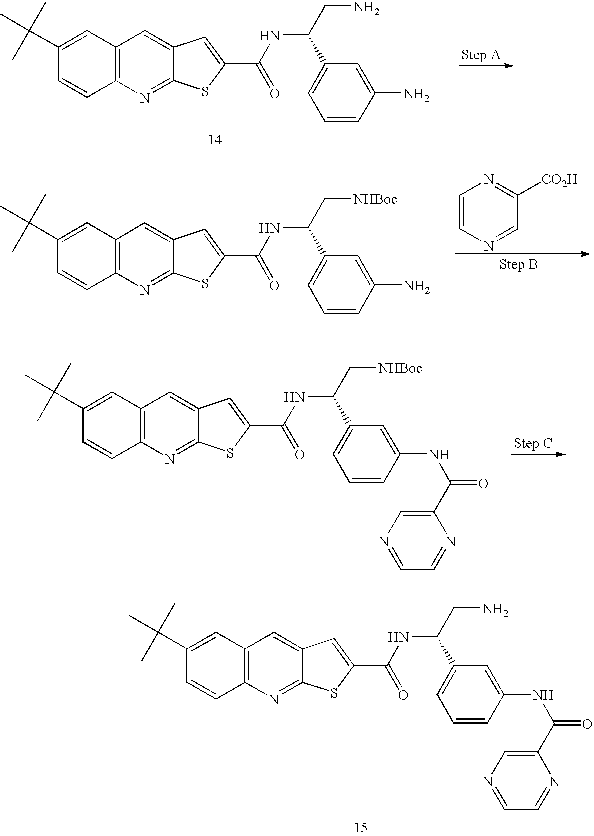 Step A:

{2(S)-(3-Amino-phenyl)-2-[(6-tert-butyl-thieno[2,3-b]quinoline-2-carbonyl)-amino]-ethyl}-carbamic acid tert-butyl ester: To a solution of 6-tert-butyl-thieno[2,3-b]quinoline-2-carboxylic acid [2(S)-amino-1-(3-amino-phenyl)-ethyl]-amide in dichloromethane (7.6 mL), was added Et₃N (154 mg, 1.53 mmol). The reaction was cooled to 0° C., and (Boc)₂O (158 mg, 0.72 mmol) was then added in one portion. The reaction was stirred from 0° C. to rt overnight. The reaction was diluted with CH₂Cl₂ (10 mL), washed with H₂O, brine, dried over Na₂SO₄. The organic layer was concentrated. The residue was purified by silica gel chromatography with 66% EtOAc/hexanes to give 347 mg of {2(S)-(3-amino-phenyl)-2-[(6-tert-butyl-thieno[2,3-b]quinoline-2-carbonyl)-amino]-ethyl}-carbamic acid tert-butyl ester (87% yield).

Step B:

(2-[(6-tert-Butyl-thieno[2,3-b]quinoline-2-carbonyl)-amino]-2(S)-{3-[(pyrazine-2-carbonyl)-amino]-phenyl)-ethyl)-carbamic acid tert-butyl ester. To a solution of (2(S)-(3-amino-phenyl)-2-[(6-tert-butyl-thieno[2,3-b]quinoline-2-carbonyl)-amino]-ethyl)-carbamic acid tert-butyl ester (19.7 mg, 0.04 mmol) in DMF (0.5 mL) was added isoxazole-5-carboxylic acid (12.8 mg, 0.11 mmol), NMM (21 μL, 0.19 mmol), followed by HATU (43 mg, 0.11 mmol). The reaction mixture was stirred at rt overnight. H₂O (10 mL) was added to the reaction, the white precipitate was collected by filtration (washing with H₂O), and dried under vacuum to give 23 mg of the product that was used directly in step C.

Step C:

6-tert-Butyl-thieno[2,3-b]quinoline-2-carboxylic acid (2-amino-1(S)-{3-[(pyrazine-2-carbonyl)-amino]-phenyl}-ethyl)-amide. To a solution (2-[(6-tert-butyl-thieno[2,3-b]quinoline-2-carbonyl)-amino]-2(S)-{3-[(pyrazine-2-carbonyl)-amino]-phenyl}-ethyl)-carbamic acid tert-butyl ester (23 mg, 0.04 mmol) in 0.2 mL/0.6 mL (TFA/CH₂Cl₂) was stirred at rt for 1.5 hr. The reaction was concentrated in vacuo, MeOH (1 mL) was added to the residue, followed by the addition of saturated Na₂CO₃ solution (20 drops). A white solid precipitated from the solution and was collected via filtration. The crude product was purified by prep TLC (10% MeOH/CH₂Cl₂) to provide 10.8 mg (55%, 2 steps) of 6-tert-butyl-thieno[2,3-b]quinoline-2-carboxylic acid (2-amino-1(S)-{3-[(pyrazine-2-carbonyl)-amino]-phenyl}-ethyl)-amide. LCMS: MH⁺=525; mp (° C.)=156-158.

### Example 16

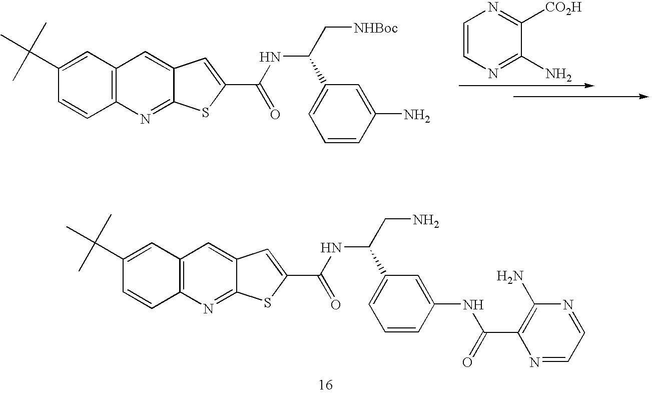

Following the same procedure set forth in Example 15, Step B-C, only substituting the acid with 3-aminopyrazine-2-carboxylic acid gave 6-tert-butyl-thieno[2,3-b]quinoline-2-carboxylic acid (2-amino-1(S)-{3-[(pyrazine-2-carbonyl)-amino]-phenyl}-ethyl)-amide. LCMS: MH⁺=540; mp (° C.)=164-167.

### Example 17

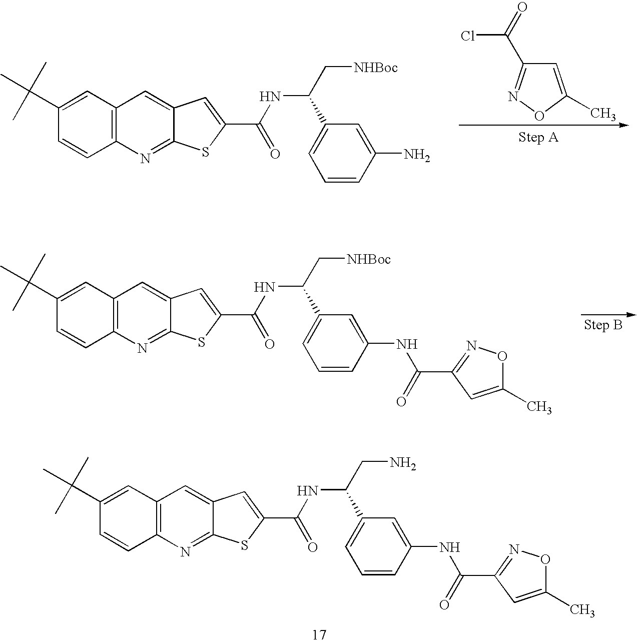 Step A:

(2-[(6-tert-Butyl-thieno[2,3-b]quinoline-2-carbonyl)-amino]-2(S)-{3-[(5-methyl-isoxazole-3-carbonyl)-amino]-phenyl}-ethyl)-carbamic acid tert-butyl ester. To a solution of {2(S)-(3-amino-phenyl)-2-[(6-tert-butyl-thieno[2,3-b]quinoline-2-carbonyl)-amino]-ethyl}-carbamic acid tert-butyl ester (52.1 mg, 0.10 mmol) in CH₂Cl₂ (1.0 mL), at 0° C. was added Et₃N (21 μL, 0.15 mmol), followed by a solution of 5-methyl-isoxazole-3-carbonyl chloride in CH₂Cl₂ (0.2 mL). The reaction mixture was allowed to slowly warm to rt and stirred under a N₂ atmosphere for 2.5 hr. The reaction was diluted with MeOH (1.5 mL) and continued stirring for 30 min. Dichloromethane (10 mL) was added and the solution was washed with 0.5 N HCl (aq.). The organic phase was dried over Na₂SO₄, filtered and concentrated to give a brown oil which was used directly in step B.

Step B:

6-tert-Butyl-thieno[2,3-b]quinoline-2-carboxylic acid (2-amino-1(S)-{3-[(5-methyl-isoxazole-3-carbonyl)-amino]-phenyl}-ethyl)-amide. A solution of (2-[(6-tert-butyl-thieno[2,3-b]quinoline-2-carbonyl)-amino]-2(S)-{3-[(5-methyl-isoxazole-3-carbonyl)-amino]-phenyl}-ethyl)-carbamic acid tert-butyl ester in TFA/CH₂Cl₂ (0.5 mL/1.0 mL) was stirred at rt for 2 hr. The reaction solution was concentrated and the residue was treated with MeOH (2 mL) and saturated Na₂CO₃ solution to pH=8. A solid precipitated from the solution and was collected by filtration. The product was purified by prep TLC (10% MeOH/CH₂Cl₂ containing 1% NH₄OH) to yield 42.2 mg (80% yield, two steps) of 6-tert-butyl-thieno[2,3-b]quinoline-2-carboxylic acid (2-amino-1(S)-{3-[(5-methyl-isoxazole-3-carbonyl)-amino]-phenyl}-ethyl)-amide.

The amine HCl salt was prepared: The product (42.2 mg) was dissolved in minimal CH₂Cl₂, and 1 equivalent of 1 N HCl/Et₂O (80.0 μL) was added while stirring rapidly. Et₂O was added and the resulting solid was collected by filtration to give 45.3 mg of HCl salt. LCMS: 528; mp (° C.)=186-193 (dec.)

### Example 18

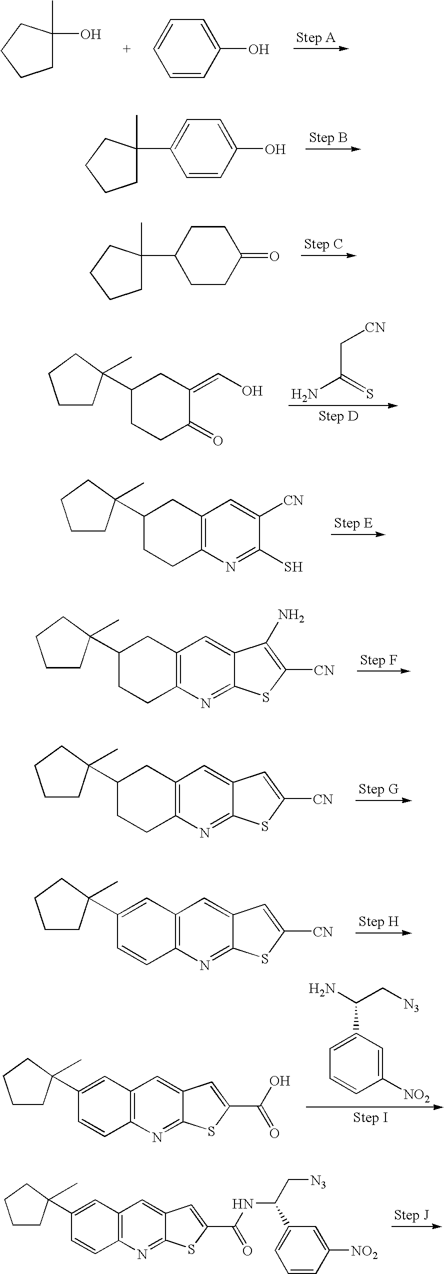 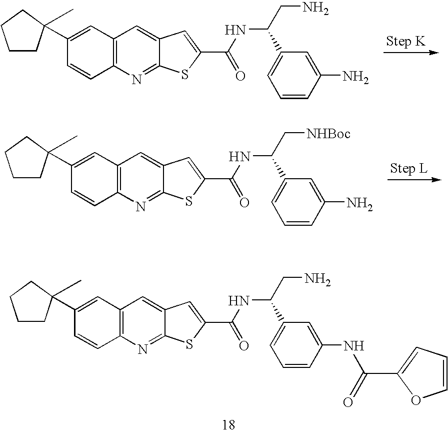 Step A:

4-(1-Methyl-cyclopentyl)phenol: A solution of phenol (1.0 g, 10.62 mmol)) in TFA (6.6 mL) at 25° C. was treated with 1-methylcyclopentanol (1.4 mL, 1.1 equiv.) followed by conc. H₂SO₄ (0.14 mL). Stirring was continued at 25° C. for 18 h. The solution was concentrated and the residue was diluted with CH₂Cl₂ (25 mL). The organic layer was washed with H₂O (50 mL), saturated NaHCO₃ (50 mL) and saturated NaCl (50 mL). The combined organic layer was dried (Na₂SO₄), filtered and concentrated under reduced pressure to yield 4-(1-methyl-cyclopentyl)-phenol.

Step B:

4-(1-Methyl-cyclopentyl)-cyclohexanone: 4-(1-Methyl-cyclopentyl)-phenol (1.0 g, 5.21 mmol) in hexanes (10 mL) and pH 7.4 phosphate buffer (10 mL) at 25° C. was treated with rhodium chloride hydrate (38% Rh w/w, 0.068g, 0.323 mmol) and tetra-n-butylammonium sulfate (0.19 g, 0.55 mmol). The solution was hydrogenated for 20 h at 60 psi. The solution was filtered through a pad of Celite. The two layers were separated. The aqueous layer was extracted with EtOAc and the combined organic layers were washed with saturated NaCl, dried (Na₂SO₄), filtered and concentrated under reduced pressure to yield 4-(1-methyl-cyclopentyl)-cyclohexanol.

A solution of Dess-Martin periodinane (1.10 equiv.) in CH₂Cl₂ at 25° C. was treated with 4-(1-methylcyclopentyl)-cyclohexanol in CH₂Cl₂. Trifluoroacetic acid (1.0 equiv.) was added and the solution was stirred 25° C. for 2 h. The solution was diluted with CH₂Cl₂ (18 mL) and Et₂O (60 mL). 1N NaOH (aqueous) was added dropwise and the mixture was stirred for 1 h and the organic layer was separated. The organic layer was washed with 1N NaOH (aqueous) and H₂O. The organic layer was dried (Na₂SO₄), filtered and concentrated under reduced pressure to give 4-(1-methyl-cyclopentyl)-cyclohexanone.

Step C:

2-Formyl-4-(1-methyl-cyclopentyl)-cyclohexanone: Sodium hydride 60% dispersion in mineral oil (1.5 equiv.) was suspended in anhydrous ether and cooled to 0° C. 4-(1-Methyl-cyclopentyl)-cyclohexanone (1.0 equiv.) and ethyl formate (1.5 equiv.) were dissolved in anhydrous ether and added to the NaH suspension. Ethanol (0.7 equiv.) was added and the reaction was stirred at 0° C. for 5 h and gradually warmed to 25° C. The suspension was extracted with H₂O, and the combined aqueous extracts were acidified to pH 3 with 4N aqueous HCl. The resulting suspension was extracted with ether, and the combined ether extracts were washed with brine, dried (Na₂SO₄), filtered and concentrated under reduced pressure to yield 2-formyl-4-(1-methyl-cyclopentyl)-cyclohexanone.

Step D:

2-Mercapto-6-(1-methyl-cyclopentyl)-5,6,7,8-tetrahydro-quinoline-3-carbonitrile: 2-Formyl-4-(1-methylcyclopentyl)-cyclohexanone was suspended in H₂O, and a solution of piperidine acetate [prepared from piperidine (3 equiv.), acetic acid (3 equiv.) and H₂O] was added, followed by 2-cyanothioacetamide (1.03 equiv.). The mixture was heated to 100° C. over 15 min., and then stirred for 40 min. at 100° C. Acetic acid was added, and the reaction mixture was slowly cooled to room temperature. The reaction was filtered and the resulting solid was dried under vacuum to give 2-mercapto-6-(1-methyl-cyclopentyl)-5,6,7,8-tetrahydro-quinoline-3-carbonitrile.

Step E:

3-Amino-6-(1-methyl-cyclopentyl)-5,6,7,8-tetrahydrothieno[2,3-b]quinoline-2-carbonitrile: The crude mercapto-nitrile was dissolved in dimethylformamide and 2-chloroacetonitrile was added. The solution was cooled to 0° C., and 20% aqueous potassium hydroxide was added. The reaction was stirred for 3 h at 0° C. to 4° C., then diluted with ice-water. After the ice had melted, the resulting suspension was filtered, and the filter residue was taken up in acetone and concentrated under reduced pressure. The residue was purified by flash chromatography to yield 3-amino-6-(1-methyl-cyclopentyl)-5,6,7,8-tetrahydrothieno[2,3-b]quinoline-2-carbonitrile.

Step F:

6-(1-Methyl-cyclopentyl)-5,6,7,8-tetrahydro-thieno[2,3-b]quinoline-2-carbonitrile: 3-Amino-6-(1-methyl-cyclopentyl)-5,6,7,8-tetrahydrothieno[2,3-b]quinoline-2-carbonitrile was added dropwise to a solution of 90% t-butylnitrite (1.5 equiv.) in DMF at 65° C. The reaction was stirred at 65° C. until complete. Upon cooling to rt, the reaction solution was added to H₂O and extracted with EtOAc. The organic phase was dried over Na₂SO₄, and concentrated. The residue was purified by flash chromatography eluting with 15% EtOAc/hexanes to give 6-(1-methyl-cyclopentyl)-5,6,7,8-tetrahydrothieno[2,3-b]quinoline-2-carbonitrile.

Step G:

6-(1-Methyl-cyclopentyl )-5,6,7,8-tetrahydrothieno[2,3-b]quinoline-2-carbonitrile: Following the same procedure set forth in Example 12, step A, only substituting the tricyclic carboxylic acid with 6-(1-methyl-cyclopentyl)-5,6,7,8-tetrahydrothieno[2,3-b]quinoline-2-carbonitrile gave 6-(1-methyl-cyclopentyl)-thieno[2,3-b]quinoline-2-carbonitrile.

Step H:

6-(1-Methyl-cyclopentyl)-thieno[2,3-b]quinoline-2-carboxylic acid: A mixture of 6-(1-methyl-cyclopentyl)-thieno[2,3-b]quinoline-2-carbonitrile in 85% phosphoric acid was stirred at 160° C. for 4 h. After it was cooled to room temperature, ice H₂O was added. The solid was collected by filtration, washed with H₂O and then dried under vacuum. The mother liquor was extracted with CH₂Cl₂. The organic phase was dried over anhydrous Na₂SO₄ and then concentrated under vacuum. The solid residue was combined with the solid from the previous filtration to give 6-(1-methyl-cyclopentyl)-thieno[2,3-b]quinoline-2-carboxylic acid.

Step I:

6-(1-Methyl-cyclopentyl)-thieno[2,3-b]quinoline-2-carboxylic acid [2-azido-1(S)-(3-nitro-phenyl)-ethyl]-amide: Following the same procedure set forth in Example 14, step A, only substituting the carboxylic acid with 6-(1-methyl-cyclopentyl)-thieno[2,3-b]quinoline-2-carboxylic acid (Step H) gave 6-(1-methyl-cyclopentyl)-thieno[2,3-b]quinoline-2-carboxylic acid [2-azido-1(S)-(3-nitro-phenyl)ethyl]-amide.

Step J:

6-(1-Methyl-cyclopentyl)thieno[2,3-b]quinoline-2-carboxylic acid [2(S)-amino-1-(3-amino-phenyl)-ethyl]-amide: Following the same procedure set forth in Example 14, step B, only substituting 6-tert-butyl-thieno[2,3-b]quinoline-2-carboxylic acid [2-azido-1(S)-(3-nitro-phenyl)-ethyl]-amide with 6-(1-methyl-cyclopentyl)-thieno[2,3-b]quinoline-2-carboxylic acid [2-azido-1(S)-(3-nitro-phenyl)-ethyl]-amide (Step I) gave 6-(1-methyl-cyclopentyl)-thieno[2,3-b]quinoline-2-carboxylic acid [2-amino-1(S)-(3-amino-phenyl)-ethyl]-amide.

Step K:

(2(S)-(3-Amino-phenyl)-2{[6-(1-methyl-cyclopentyl)thieno[2,3-b]quinoline-2-carbonyl]-amino}-ethyl)-carbamic acid tert-butyl ester: Following the same procedure set forth in Example 15, step A, only substituting 14 with 6-(1-methyl-cyclopentyl)-thieno[2,3-b]quinoline-2-carboxylic acid [2-amino-1(S)-(3-amino-phenyl)-ethyl]-amide (Step J) gave (2(S)-(3-amino-phenyl)-2-{[6-(1-methyl-cyclopentyl)-thieno[2,3-b]quinoline-2-carbonyl]-amino}-ethyl)-carbamic acid tert-butyl ester. LC-MS: MH⁺=545.3.

Step L:

6-(1-Methyl-cyclopentyl)-thieno[2,3-b]quinoline-2-carboxylic acid (2-amino-1(S)-{3-[(furan-2-carbonyl)-amino]-phenyl}-ethyl)-amide (18): Following the same procedure set forth in Example 17, steps A-B, only substituting 14A with (2(S)-(3-amino-phenyl)-2-{[6-(1-methyl-cyclopentyl)-thieno[2,3-b]quinoline-2-carbonyl]-amino}-ethyl)-carbamic acid tert-butyl ester (Step K) and substituting the acid chloride with 2-furanyl carbonyl chloride gave 6-(1-methyl-cyclopentyl)-thieno[2,3-b]quinoline-2-carboxylic acid (2-amino-1(S)-{3-[(furan-2-carbonyl)-amino]-phenyl}-ethyl)-amide (18). LC-MS: MH⁺=539.3.

### Example 19

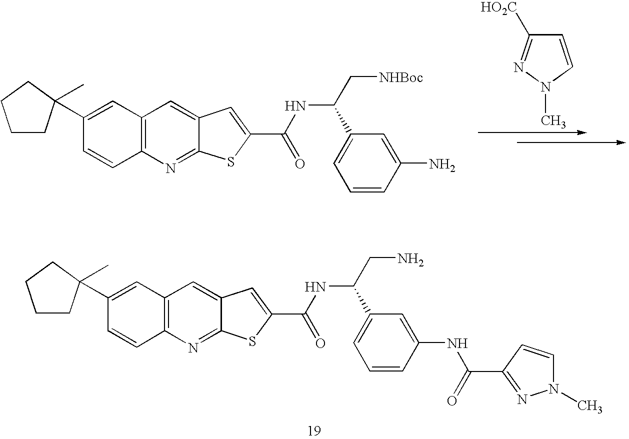

6-(1 -Methyl-cyclopentyl)-thieno[2,3-b]quinoline-2-carboxylic acid (2-amino-1(S)-{3-[(1-methyl-1H-pyrazole-3-carbonyl)amino]-phenyl}-ethyl)-amide(19): Following the same procedure set forth in Example 15, Steps B-C, only substituting 14A with (2(S)-(3-amino-phenyl)-2-{[6-(1-methyl-cyclopentyl)-thieno[2,3-b]quinoline-2-carbonyl]-amino}-ethyl)-carbamic acid tert-butyl ester and substituting the carboxylic acid with 1-methyl-1H-pyrazole-3-carboxylic acid gave 6-(1-methyl-cyclopentyl)-thieno[2,3-b]quinoline-2-carboxylic acid (2-amino-1(S)-{3-[(1-methyl-1H-pyrazole-3-carbonyl)-amino]-phenyl}-ethyl)-amide (19). LCMS: MH⁺=553.3.

KSP Assays:

Endpoint Assay:

Serial dilutions of the compounds were prepared in a low binding, 96-well microtiter plate (Costar #3600) using 40% DMSO (Fisher BP231). The diluted compounds were added to a 384-well microtiter plate (Fisher 12-565-506). The following was then added to each well of the 384 microtiter plate: 55 μg/mL purified microtubules (Cytoskeleton TL238), 2.5-10 nM KSP motor domain (made according to Hopkins et al, Biochemistry, (2000) 39, 2805-2814)), 20 mM ACES pH 7.0 (Sigma A-7949), 1 mM EGTA (Sigma E-3889), 1 mM MgCl₂ (Sigma M-2670), 25 mM KCl (Sigma P-9333), 10 μM paclitaxel (Cytoskeleton TXD01), and 1 mM DTT (Sigma D5545) (final concentration). Following a 10 minute incubation, ATP (Sigma A-3377) (final concentration of ATP: 100 μM) was added to start the reaction. The final reaction volume was 25 μL. Final test compound concentration ranged from 50 μM to 5 nM, and 10 μM to 0.128 nM. The reaction was incubated for 1 hour at room temperature. The reaction was stopped by the addition of 50 μL Biomol green reagent (Biomol AK111) per well, and was allowed to incubate for 20 minutes at room temperature. The 384-well microtiter plate was then transferred to an absorbance reader (Molecular Devices SpectraMax plus) and a single measurement was taken at 620 nm.

Kinetic Assay:

Compound dilutions were prepared as described previously. 25A25 buffer consisted of the following: 25 mM ACES pH 6.9, 2 mM MgOAc (Sigma M-9147), 2 mM EGTA, 0.1 mM EDTA (Gibco 144475-038), 25 mM KCl, 1 mM 2-mercaptoethanol (Biorad 161-0710), 10 μM paclitaxel, and 0.5 mM DTT. Solution 1 consisted of the following: 3.75 mM (final concentration) phosphoenol pyruvic acid (PEP, 2.5×) (Sigma P-7127), 0.75 mM MgATP (2.5×) (Sigma A-9187) in 1×25A25 buffer. Solution 2 consisted of the following: 100-500 nM KSP motor domain (2×), 6 U/mL pyruvate kinase/lactate dehydrogenase (2×) (Sigma P-0294), 110 μg/mL purified microtubules (2×), 1.6 μM β-nicotinamide adenine di-nucleotide, reduced form (NADH, 2×) (Sigma N-8129) in 1×25A25 buffer. Compound dilutions [8] were added to a 96-well microtiter plate (Costar 9018), and 40 μL of solution 1 was added to each well. The reaction was started by adding 50 μL of solution 2 to each well. The respective final assay concentrations were: 1.5 mM PEP, 0.3 mM MgATP, 50-250 nM KSP motor domain, 3 U/mL pyruvate kinase/lactate dehydrogenase, 55 μg/mL purified microtubules, 0.8 μM NADH (final concentrate). The microtiter plate was then transferred to an absorbance reader and multiple readings were taken for each well in a kinetic mode at 340 nm (25 measurements for each well approximately every 12 seconds, spread approximately over about 5 minutes time span). For each reaction, a rate of change was determined.

Calculations:

For both endpoint and kinetic assays, the percent activity for each concentration is calculated using the following equation:
Y=((X−background)/(positive control−background))*100

Y is the % activity and X is the measured reading (OD620 or rate)

For an IC₅₀ determination, the % activity was fit by the following equation using a nonlinear curve-fitting program for sigmoidal dose-responses (variable slopes) (GraphPad Prizm).
Y=Bottom+(Top−Bottom)/(1+10ˆ((Log EC50−X)*HillSlope))

X is the logarithm of concentration. Y is the response.

Y starts at Bottom and goes to Top with a sigmoid shape.

KSP inhibitory activities (endpoint assay) for representative compounds are shown in Table 1 below. All IC₅₀ values are obtained from the end point assay.

TABLE 1

| | IC₅₀ | | IC₅₀ | | IC₅₀ |
|---|---|---|---|---|---|
| COMD | (μM) | COMD | (μM) | COMD | (μM) |
| 1 | 0.16 | 2 | 0.08 | 3 | >10 |
| 4 | >10 | 5 | 0.07 | 6 | 0.29 |
| 7 | >10 | 8 | 9.0 | 9 | 0.6 |
| 10 | >10 | 11 | 8.4 | 12 | 0.038 |
| 13 | 0.035 | 14 | 0.025 | 15 | 0.030 |
| 16 | 0.026 | 17 | 0.036 | | |

### REFERENCES

KSP/Kinesin as Target

1) Blangy, A et al. (1995) Cell 83, 1159-1169 (cloning of human KSP, function in mitosis).
2) Sawin, K. and Mitchison, T. J. (1995) Proc. Natl. Acad. Sci. 92, 4289-4293 (Xenopus Egd5, conserved motor domain, function).
3) Huang, T.-G. and Hackney, D. D. (1994) J. Biol. Chem. 269,16493-16501 (Drosphila kinesin minimal motor domain definition, expression and purification from E. coli).
4) Kaiser A. et al. (1999) J. Biol. Chem. 274, 18925-18931 (overexpression of KSP motor domain, function in mitosis, inhibition of growth by targeting KSP).
5) Kapoor T. M and Mitchison, T. J. (1999) Proc. Natl. Acad. Sci. 96, 9106-9111 (use of KSP motor domain, inhibitors thereof).
6) Mayer, T. U. (1999) Science 286, 971-974 (KSP inhibitors as anticancer drugs).
   KSP Assays (Endpoint and Kinetics)
7) Wohlke, G. et al. (1997) Cell 90, 207-216 (expression and purification of kinesin motor domain, kinetics assay, endpoint assay).
8) Geladeopoulos, T. P. et al. (1991) Anal. Biochem. 192, 112-116 (basis for endpoint assay).
9) Sakowicz, R. et al. (1998) Science 280, 292-295 (kinetics assay).
10) Hopkins, S. C. et al. (2000) Biochemistry 39, 2805-2814 (endpoint and kinetics assay).
11) Maliga, Z. et al. (2002) Chem. & Biol. 9, 989-996 (kinetics assay).

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A compound represented by the structural Formula I: 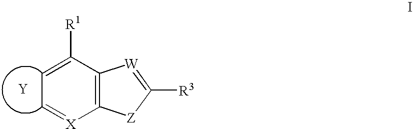
or a pharmaceutically acceptable salt, solvate or ester thereof, wherein:
ring Y is a 5- to 6-membered aryl or a 5- or 6-membered heteroaryl fused as shown in Formula I, wherein in said aryl and heteroaryl each substitutable ring carbon is independently substituted with R² and each substitutable ring nitrogen is independently substituted with R⁶;
W is N or C(R¹²);
X is N or N-oxide;
Z is S, S(═O) or S(═O)₂;
R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹;
each R² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴ R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
each R³ is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S )NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴R⁵, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
each R⁴ and R⁵ is independently selected from the group consisting of H, alkyl, cycloalkyl, cycloalkylalkyl, heterocyclyl, heterocyclylalkyl, aryl, aralkyl, heteroaryl, heteroaralkyl, —OR⁷, —C(O)R⁷, and —C(O)OR⁷, wherein each of said alkyl, cycloalkyl, cycloalkylalkyl, heterocyclyl, heterocyclylalkyl, aryl, aralkyl, heteroaryl, or heteroaralkyl is optionally substituted with 14 R⁸ moieties;
or R⁴ and R⁵, when attached to the same nitrogen atom, are optionally taken together with the nitrogen atom to which they are attached to form a 3-6 membered heterocyclic ring having 0-2 additional heteroatoms selected from N, O or S;
each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;
each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;
each R⁸ is independently selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹, wherein each of said alkyl, cycloalkyl, heteroacyclyl, aryl, and heteroaryl is optionally independently substituted with 1-3 moieties selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)OR¹⁰, —C(O)NR¹⁰R¹¹, —CF³, —OCF₃, —NR¹⁰C(O)OR¹¹, and —NR¹⁰C(O)R⁴⁰;
or two R⁸ groups, when attached to the same carbon atom, are optionally taken together with the carbon atom to which they are attached to form a C═O or a C═S group;
each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;
each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;
each R¹¹ is independently H or alkyl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S;
each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties; and
R⁴⁰ is selected from the group consisting of cycloalkyl, heterocyclyl, aryl and heteroaryl, wherein each of said cycloalkyl, heterocyclyl, aryl and heteroaryl are optionally independently substituted with 1-3 moieties independently selected from the group consisting of —CN, —OH, halo, alkyl, haloalkyl, alkoxy, and —NR¹⁰R¹¹;
with the proviso that the compound of Formula I excludes any one of the following: 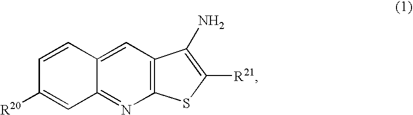
wherein R²⁰ is H, —CH₃ or —OCH₃ and R²¹ is —C(O)CH₃, —C(O)CH═CH-phenyl or —C(O)CH═CH-(4-methoxyphenyl); 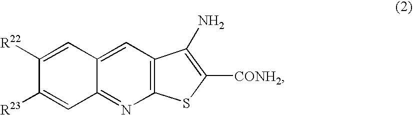
wherein R²² and R²³ are independently H or methoxy; 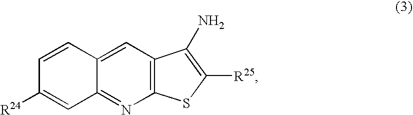
wherein R²⁴ is methyl, methoxy or —Cl and R²⁵ is —CONH₂ or —CO₂Et; 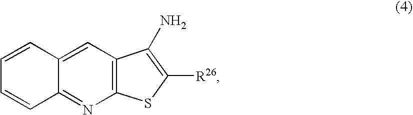
wherein R²⁶ is —CO₂Me, —CO₂Et, —CO₂H, —C(O)-phenyl, —C(O)-p-methylphenyl, —C(O)-p-bromophenyl, —C(O)CH₃, —CN, —C(O)NH-phenyl, —C(O)NH-p-methoxyphenyl, —C(O)NHNH₂, —C(O)NH-p-chlorophenyl, 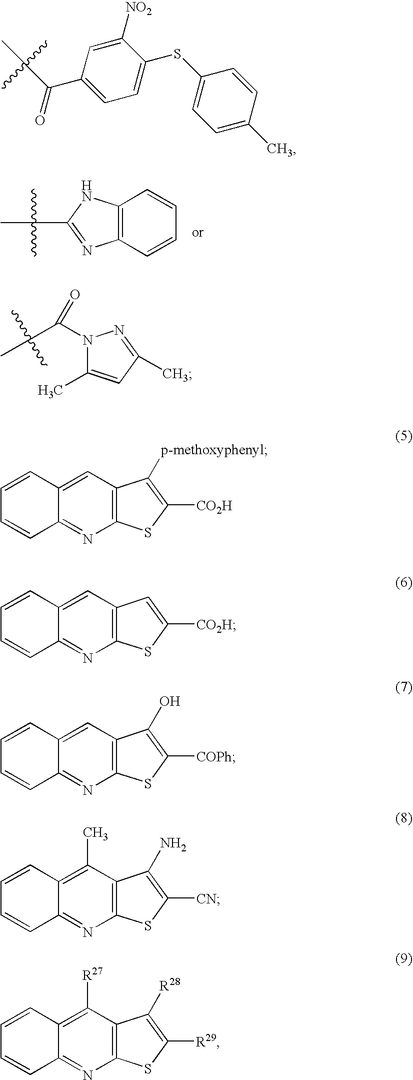
wherein:
R²⁷ is H, —OH, —OCH₃ or —OCH(CH₃)₂,
R²⁸ is —OH, —OCH₂CN or —OC(O)NH(CH₂)₅CN, and
R²⁹ is —C(O)OCH(CH₃)₂ or —C(O)O-cyclohexyl; 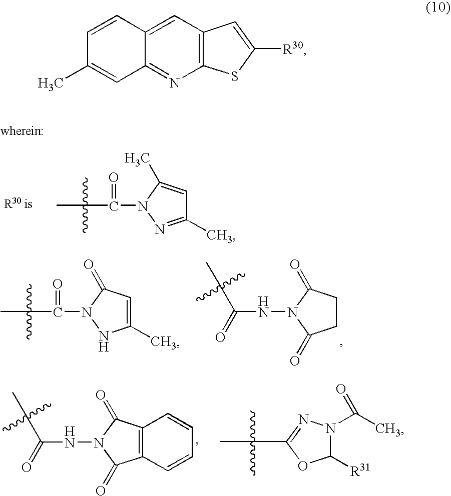
—CO₂CH₃, —CO₂C₂H₅, —C(O)NH₂, —C(O)NHNH₂, or —C(O)NHCH₃ and R³¹ is C₆H₅, p-OHC₆H₄ or p-CH₃C₆H₄; 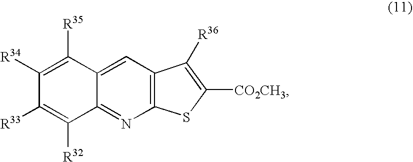
wherein:
R³² is H or NO₂,
R³³ and R³⁴ are independently H, —OCH₃ or —OC₂H₅,
R³⁵ is H or —OCH₃, and
R³⁶ is H, CH₃ or C₆H₅; 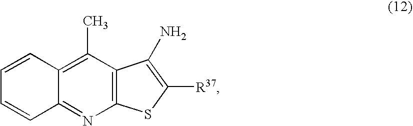
wherein:
R³⁷ is —CO₂Me, —CO₂Et, —CO₂H, —C(O)NH₂, —C(O)NHNH₂, —CN, —C(O)NH-p-methoxyphenyl, —C(O)NH-(2-pyridyl) or 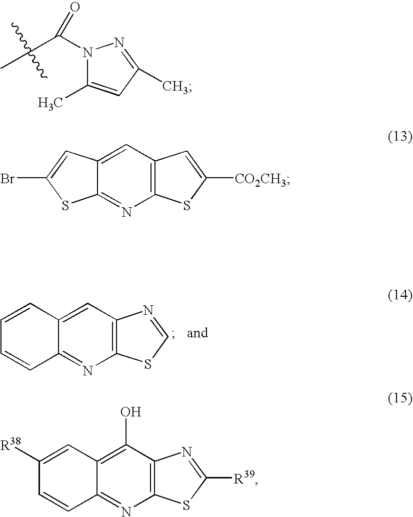
wherein R³⁸ is H, methyl or CF₃ and R³⁹ is SMe, SOMe, SO₂Me, Cl, NH(CH₂)NEt₂, or N-(N′-methyl)piperazinyl.

2. The compound of claim 1 represented by Formula II:

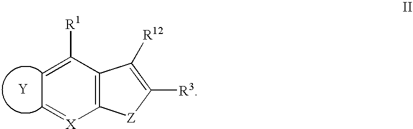

3. The compound of claim 1 represented by Formula III:

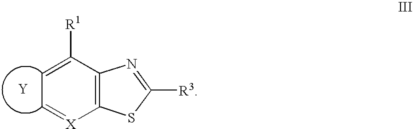

4. The compound of claim 2, wherein X is N.

5. The compound of claim 2, wherein X is N-oxide.

6. The compound of claim 2, wherein Z is S.

7. The compound of claim 2, wherein Z is S(═O).

8. The compound of claim 2, wherein Z is S(═O)₂.

9. The compound of claim 2, wherein ring Y is benzo wherein each substitutable ring carbon is independently substituted with R².

10. The compound of claim 9, wherein R² is H, alkyl, aryl, aralkyl, cycloalkyl, cycloalkylalkyl, —CF₃, alkylsilyl, alkoxy or —NR⁴R⁵.

11. The compound of claim 1, wherein R⁶ is H, alkyl, aralkyl, haloalkyl, cycloalkylalkyl or —C(O)OR⁷ wherein R⁷ is alkyl.

12. The compound of claim 1, wherein R¹² is H, halo, —NR⁴R⁵ or —OR⁷.

13. The compound of claim 1, wherein R³ is H, alkyl, heterocyclyl, heteroaryl, —(CR¹⁰R¹¹)₁₋₆—OR⁷, —C(O)R⁴, —C(O)OR⁷, —C(O)NR⁴R⁵, —C(S)NR⁴R⁵, —C(O)NR⁴OR⁷, —C(O)NR⁷NR⁴R⁵, —NR⁴R⁵, —N(R⁴)C(O)R⁵, —N(R⁴)C(O)NR⁴R⁵, —(CR¹⁰R¹¹)₀₋₆SR⁷, SO₂R⁷, —SO₂NR⁴R⁵, —CN, —C(═NR⁷)NR⁴R⁵, —C(O)NR⁷(CH₂)₁₋₁₀NR⁴R⁵, or —C(O)NR⁷(CH₂)₁₋₁₀OR⁷, wherein said alkyl, heterocyclyl or heteroaryl is optionally substituted with 1-3 R⁹ moieties.

14. The compound of claim 1, wherein R¹ is H, halo, —S-alkyl, alkoxy or hydroxy.

15. The compound of claim 14, wherein R¹ is H, Cl, OH or —SCH₃.

16. The compound of claim 2, represented by Formula II-a:

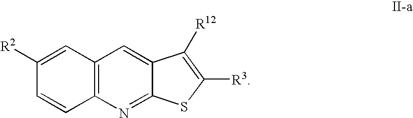

17. The compound of claim 16, wherein:
R² is alkyl, aryl, aralkyl, cycloalkyl, cycloalkylalkyl, —CF₃, alkylsilyl, or —NR⁴R⁵;
R³ is H, heterocyclyl, heteroaryl, —C(O)OR⁷, —C(O)R⁴, —C(O)NR⁴R⁵, —C(S)NR⁴R⁵, —C(O)N(R⁴)OR⁷, —NR⁴R⁵, —NR⁴C(O)R⁵, —NR⁴C(O)NR⁴R⁵, —SO₂R⁷, —SO₂NR⁴R⁵, —CN, —(CR¹⁰R¹¹)₁₋₆SR⁷, or —C(═NR⁷)NR⁴R⁵; and
R¹² is H, halo, —NR⁴R⁵, or —OR⁷.

18. The compound of claim 17, wherein:
R² is alkyl or alkylsilyl; wherein said alkyl is C₁-C₆ alkyl and said alkylysilyl is C₁-C₆ alkylsilyl;
R³ is —CN, —C(O)NR⁴R⁵, —C(O)R⁴, —C(S)NR⁴R⁵, —C(═NR⁷)NR⁴R⁵, heterocyclyl, —C(O)OR⁷, —C(O)N(R⁴)OR⁷, —SO₂R⁷, —SO₂NR⁴R⁵, —N(R⁴)C(O)R⁵, or —N(R⁴)C(O)NR⁴R⁵; wherein said —C(O)NR⁴R⁵ is —C(O)N(R⁶¹)₂, said —C(O)R⁴ is —C(O)R⁶², said —C(S)NR⁴R⁵ is —C(S)N(R⁶⁰)₂, said —C(═NR⁷)NR⁴R⁵ is —C(═NR⁶⁰)N(R⁶⁰)₂, said heterocyclyl is tetrazolyl, said —C(O)OR⁷ is —C(O)OR⁶¹, said —C(O)N(R⁴)OR⁷ is —C(O)N(R⁶⁰)OR⁶⁰, said —SO₂R⁷ is —SO₂R⁶⁰, said —SO₂NR⁴R⁵ is —SO₂N(R⁶⁰), said —N(R⁴)C(O)R⁵ is —N(R⁶⁰)C(O)R⁶⁰, and said —N(R⁴)C(O)NR⁴R⁵ is —N(R⁶⁰)C(O)N(R⁶⁰)₂;
R¹² is H, halo, —NR⁴R⁵, or —OR⁷; wherein said —NR⁴R⁵ is —N(R⁶⁰)₂, and said —OR⁷ is —OR⁶⁰;
each R⁶⁰ independently is H or C₁-C₆ alkyl;
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰; and
R⁶² is N-pyrrolidinyl, N-piperidinyl, N-piperazinyl, N,N′-methylpiperazinyl; wherein each member of R⁶² is optionally substituted with —OR⁶⁰, —CO₂R⁶⁰, or —N(R⁶⁰)₂; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

19. The compound of claim 18, wherein:
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

20. The compound of claim 18, wherein:
R² is C₁-C₆ alkylsilyl;
R³ is —C(O)NR⁴R⁵ wherein said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

21. The compound of claim 19, wherein:
R² is C₁-C₆ alkyl; and
R³ is —CN, —C(O)N(R⁶¹)₂ or —C(O)OR⁶¹; wherein said —C(O)N(R⁶¹)₂ is —C(O)N(R⁶³)₂, and said —C(O)OR⁶¹ is —C(O)OR⁶⁰; and
R⁶³ is H, C₁-C₆ alkyl or phenyl, wherein said C₁-C₆ alkyl is optionally substituted with —N(R⁶⁰)C(O)R⁶⁰ or —N(R⁶⁰)₂, and said phenyl is is optionally substituted with 1-2 moieties independently selected from the group consisting of —N(R⁶⁰)₂ and —N(R⁶⁰)C(O)R⁷⁰.

22. The compound of claim 16, wherein R¹² is H.

23. The compound of claim 16, wherein:
R² is alkyl;
R³ is —C(O)NR⁴R⁵;
R⁴ and R⁵ are independently selected from the group consisting of H and alkyl, wherein said alkyl is optionally substituted with 1-4 R⁸ moieties;
each R⁸ is independently selected from the group consisting of —NR¹⁰R¹¹ and aryl; wherein said aryl is optionally substituted with 1-3 moieties independently selected from the group consisting of alkyl, —NR¹⁰R¹¹ and —NR¹⁰C(O)R⁴⁰;
each R¹⁰ is independently H or alkyl;
each R¹¹ is independently H or alkyl;
R¹² is H; and
R⁴⁰ is selected from the group consisting of aryl and heteroaryl, wherein said aryl and heteroaryl are optionally independently substituted with 1-3 moieties independently selected from the group consisting of —CN, —OH, halo, alkyl, haloalkyl, alkoxy, and —NR¹⁰R¹¹.

24. The compound of claim 23, wherein said R⁸ aryl is phenyl.

25. The compound of claim 23, wherein said R⁴⁰ heteroaryl is selected from the group consisting of furanyl, pyrazolyl, pyrazinyl, oxazolyl, and isoxazolyl, each of which is optionally substituted.

26. The compound of claim 1 represented by Formula II-b: 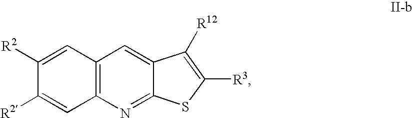
wherein R^{2′} is selected from the members of R², and wherein R^{2′} and R² can be the same of different.

27. The compound of claim 26, wherein:
R² is alkyl, aryl, aralkyl, cycloalkyl, cycloalkylalkyl, —CF₃, alkylsilyl, or —NR⁴R⁵;
R^{2′} is alkyl, aryl, aralkyl, cycloalkyl, cycloalkylalkyl, —CF₃, alkylsilyl, or —NR⁴R⁵;
R³ is H, heterocyclyl, heteroaryl, —C(O)R⁴, —C(O)OR⁷, —C(O)NR⁴R⁵, —C(S)NR⁴R⁵, —C(O)N(R⁴)OR⁷, —NR⁴R⁵, —N(R⁴)C(O)R⁵, —N(R⁴)C(O)NR⁴R⁵, —SO₂R⁷, —SO₂NR⁴R⁵, —CN, —(CR¹⁰R¹¹)₁₋₆SR⁷, or —C(═NR⁷)NR⁴R⁵; and
R¹² is H, halo, —NR⁴R⁵, or —OR⁷.

28. The compound of claim 26, wherein:
R² is alkyl or alkylsilyl; wherein said alkyl is C₁-C₆ alkyl, and said alkylsilyl is C₁-C₆ alkylsilyl;
R^{2′} is alkyl or alkylsilyl; wherein said alkyl is C₁-C₆ alkyl, and said alkylsilyl is C₁-C₆ alkylsilyl;
R³ is —CN, —C(O)NR⁴R⁵, —C(O)R⁴, —C(S)NR⁴R⁵, —C(═NR⁷)NR⁴R⁵, heterocyclyl, —C(O)OR⁷, —C(O)N(R⁴)OR⁷, —SO₂R⁷, —SO₂NR⁴R⁵, —N(R⁴)C(O)R⁵, or —N(R⁴)C(O)NR⁴R⁵; wherein said —C(O)NR⁴R⁵ is —C(O)N(R⁶¹)₂, said —C(O)R⁴ is —C(O)R⁶², said —C(S)NR⁴R⁵ is —C(S)N(R⁶⁰)₂, said —C(═NR⁷)NR⁴R⁵ is —C(═NR⁶⁰)N(R⁶⁰)₂, said heterocyclyl is tetrazolyl, said —C(O)OR⁷ is —C(O)OR⁶¹, said —C(O)N(R⁴)OR⁷ is —C(O)N(R⁶⁰)OR⁶⁰, said —SO₂R⁷ is —SO₂R⁶⁰, said —SO₂NR⁴R⁵ is —SO₂N(R⁶⁰)₂, said —N(R⁴)C(O)R⁵ is —N(R⁶⁰)C(O)R⁶⁰, and said —N(R⁴)C(O)NR⁴R⁵ is —N(R⁶⁰)C(O)N(R⁶⁰)₂;
R¹² is H, halo, —NR⁴R⁵, or —OR⁷; wherein said —NR⁴R⁵ is —N(R⁶⁰)₂, and said —OR⁷ is —OR⁶⁰;
each R⁶⁰ independently is H or C₁-C₆ alkyl;
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
R⁶² is N-pyrrolidinyl, N-piperidinyl, N-piperazinyl, N,N′-methylpiperazinyl; wherein each member of R⁶² is optionally substituted with —OR⁶⁰, —CO₂R⁶⁰, or —N(R⁶⁰)₂; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

29. The compound of claim 26, wherein said C₁-C₆ alkylsilyl in said R² and R³ is (C₁-C₆ alkyl)₃silyl.

30. The compound of claim 25, wherein R¹² is H.

31. The compound of claim 28, wherein said 5- to 6-membered heterocyclyl in R⁶¹ is morpholinyl, piperidinyl, pyrrolidinyl, or piperazinyl.

32. The compound of claim 26, wherein:
R² and R^{2′} are independently alkyl; wherein said alkyl is C₁-C₆ alkyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

33. The compound of claim 26, wherein:
R² and R^{2′} are independently C₁-C₆ alkylsilyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

34. The compound of claim 1, wherein:
R² is alkyl, said alkyl being t-butyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

35. The compound of claim 34, wherein R¹² is H.

36. The compound of claim 26, wherein:
R² is alkyl, said alkyl being t-butyl or i-propyl;
R^{2′} is alkyl, said alkyl being methyl or ethyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

37. The compound of claim 33, wherein:
R³ is —CN, —C(O)OR⁶¹ or —C(O)NR⁴R⁵; wherein said —C(O)OR⁶¹ is —C(O)OR⁶⁰, and said —C(O)NR⁴R⁵ is —C(O)N(R⁶³)₂; and
each R⁶³ independently is H or C₁-C₆ alkyl wherein said C₁-C₆ alkyl of said R⁶³ is optionally substituted with —N(R⁶⁰)C(O)R⁶⁰ or —N(R⁶⁰)₂; wherein each R⁶⁰ independently is H or C₁-C₆ alkyl.

38. The compound of claim 36, wherein R¹² is H.

39. The compound of claim 3 represented by Formula III-a: 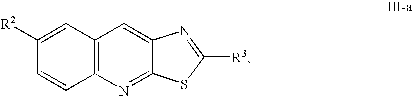
wherein:
R² is alkyl, aryl, aralkyl, cycloalkyl, cycloalkylalkyl, —CF₃, alkylsilyl, alkoxy or —NR⁴R⁵; and
R³ is H, heterocyclyl, heteroaryl, —C(O)OR⁷, —C(O)NR⁴R⁵, —C(S)NR⁴R⁵, —C(O)NR⁴OR⁷, —NR⁴R⁵, —N(R⁴)C(O)R⁵, —N(R⁴)C(O)NR⁴R⁵, —SO₂R⁷, —SO₂NR⁴R⁵, —CN, —(CR¹⁰R¹¹)₀₋₆SR⁷, or —C(═NR⁷)NR⁴R⁵.

40. The compound of claim 39, wherein R³ is —C(O)OR⁷, —C(O)NR⁴R⁵, —NR⁴R⁵, —NR⁴C(O)R⁵, —NR⁴C(O)NR⁴R⁵, —(CR¹⁰OR¹¹)₀₋₆SR⁷, or —CN.

41. The compound of claim 36, wherein:
R² is alkyl; wherein said alkyl is C₁-C₆ alkyl;
R³ is —CN, —C(O)OR⁷, —(CR¹⁰R¹¹)₀₋₆SR⁷, —C(O)NR⁴R⁵, —N(R⁴)C(O)NR⁴R⁵, —NR⁴R⁵, and —N(R⁴)C(O)R⁵; wherein said —C(O)OR⁷ is
—C(O)OR⁶⁰, said —(CR¹⁰R¹¹)₀₋₆SR⁷ is —SR⁶⁰, said —C(O)NR⁴R⁵ is C(O)N(R⁶⁰)₂, said —N(R⁴)C(O)NR⁴R⁵ ᵢₛ —NR⁶⁰C(O)N(R⁶⁰)₂, said —NR⁴R⁵ is —N(R⁶⁰)₂, and said —N(R⁴)C(O)R⁵ is —NR⁶⁰C(O)R⁶⁰; and
each R⁶⁰ is H or C₁-C₆ alkyl.

42. The compound of claim 36, wherein:
R² is alkyl or alkylsilyl; wherein said alkyl is C₁-C₆ alkyl, and said alkylsilyl is C₁-C₆ alkylsilyl;
R³ is —CN, —C(O)OR⁷, —C(O)R⁷, —C(O)NR⁴R⁵, —C(S)NR⁴R⁵, —C(═NR⁷)NR⁴R⁵, heterocyclyl, —C(O)N(R⁴)OR⁷, —SO₂R⁷, S(O)₁₋₂NR⁴R⁵, —NR⁴C(O)R⁵ or —NR⁴C(O)NR⁴R⁵; wherein said —C(O)OR⁷ is —C(O)OR⁶¹, said
—C(O)R¹ is —C(O)R⁶², said —C(O)NR⁴R⁵ is —C(O)N(R⁶¹)₂, said —C(S)NR⁴R⁵ is —C(S)N(R⁶⁰)₂, said —C(═NR⁷)NR⁴R⁵ is —C(═NR⁶⁰)N(R⁶⁰)₂, said heterocyclic is tetrazolyl, said —C(O)N(R⁴)OR⁷ is —C(O)N(R⁶⁰)OR⁶⁰, said —SO₂R⁷ is —SO₂R⁶⁰, said S(O)₁₋₂NR⁴R⁵ is —SO₂N(R⁶⁰)₂, said —NR⁴C(O)R⁵ is —N(R⁶⁰)C(O)R⁶⁰, and said —NR⁴C(O)NR⁴R⁵ is —N(R⁶⁰)C(O)N(R⁶⁰)₂;
each R60 independently is H or C₁-C₆ alkyl;
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
R⁶² is N-pyrrolidinyl, N-piperidinyl, N-piperazinyl, N,N′-methylpiperazinyl; wherein each member of R⁶² is optionally substituted with —OR⁶⁰, —CO₂R⁶⁰, or —N(R⁶⁰)₂; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

43. The compound of claim 39, wherein:
R² is alkyl; wherein said alkyl is C₁-C₆ alkyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, -N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

44. The compound of claim 39, wherein:
R² is C₁-C₆ alkylsilyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

45. The compound of claim 3 represented by Formula III-b: 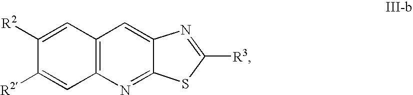
wherein:
R² is alkyl, aryl, aralkyl, cycloalkyl, cycloalkylalkyl, —CF₃, alkylsilyl, alkoxy or —NR⁴R⁵;
R^{2′} is alkyl, aryl, aralkyl, cycloalkyl, cycloalkylalkyl, —CF₃, alkylsilyl, alkoxy or —NR⁴R⁵; and
R³ is H, heterocyclyl, heteroaryl, —C(O)OR⁷, —C(O)NR⁴R⁵, —C(S)NR⁴R⁵, —C(O)NR⁴OR⁷, —NR⁴R⁵, —NR⁴C(O)R⁵, —NR⁴C(O)NR⁴R⁵, —SO₂R⁷, —SO₂NR⁴R⁵, —CN, —(CR¹⁰R¹¹)₀₋₆SR⁷, or —C(═NR⁷)NR⁴R⁵.

46. The compound of claim 45, wherein R³ is —C(O)NR⁴R⁵, —NR⁴R⁵, —NR⁴C(O)R⁵, —NR⁴C(O)NR⁴R⁵, —(CR¹⁰R¹¹)₀₋₆SR⁷, or —CN.

47. The compound of claim 45, wherein:
R² and R^{2′} are independently alkyl; wherein said alkyl is C₁-C₆-alkyl;
R³ is —CN, —(CR¹⁰R¹¹)₀₋₆SR⁷, —C(O)NR⁴R⁵, —NR⁴C(O)NR⁴R⁵, —NR⁴R⁵, or —NR⁴C(O)R⁵; wherein said —(CR¹⁰R¹¹)₀₋₆SR⁷ is —SR⁶⁰, said —C(O)NR⁴R⁵ is —C(O)N(R⁶⁰)₂, said —NR⁴C(O)NR⁴R⁵ is —NR⁶⁰C(O)N(R⁶⁰)₂, said —NR⁴R⁵ is —N(R⁶⁰)₂, and said —NR⁴C(O)R⁵ is —NR⁶⁰C(O)R⁶⁰; and
each R⁶⁰ independently is H or C₁-C₆ alkyl.

48. The compound of claim 45, wherein:
R² is alkyl or alkylsilyl; wherein said alkyl is C₁-C₆ alkyl and said alkylsilyl is C₁-C₆ alkylsilyl;
R^{2′} is alkyl or alkylsilyl; wherein said alkyl is C₁-C₆ alkyl and said alkylsilyl is C₁-C₆ alkylsilyl;
R³ is —CN, —C(O)OR⁷, —C(O)R⁷, —C(O)NR⁴R⁵, —C(S)NR⁴R⁵, —C(═N R⁷)NR⁴R⁵, heterocyclyl, —C(O)N(R⁴)OR⁷, —SO₂R⁷, S(O)₁₋₂NR⁴R⁵, —NR⁴C(O)R⁵ or —NR⁴C(O)NR⁴R⁵; wherein said —C(O)OR⁷ is —C(O)OR⁶¹, said
—C(O)R⁷ is —C(O)R⁶², said —C(O)NR⁴R⁵ is —C(O)N(R⁶¹)₂, said —C(S)NR⁴R⁵ is —C(S)N(R⁶⁰)₂, said —C(═NR⁷)NR⁴R⁵ is —C(═NR⁶⁰)N(R⁶⁰)₂, said heterocyclic is tetrazolyl, said —C(O)N(R⁴)OR⁷ is —C(O)N(R⁶⁰)OR⁶⁰, said —SO₂R⁷ is —SO₂R⁶⁰, said S(O)₁₋₂NR⁴R⁵ is —SO₂N(R⁶⁰)₂, said —NR⁴C(O)R⁵ is —N(R⁶⁰)C(O)R⁶⁰, and said —NR⁴C(O)NR⁴R⁵ is —N(R⁶⁰)C(O)N(R⁶⁰)₂;
each R⁶⁰ independently is H or C₁-C₆ alkyl;
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
R⁶² is N-pyrrolidinyl, N-piperidinyl, N-piperazinyl, N,N′-methylpiperazinyl; wherein each member of R⁶² is optionally substituted with —OR⁶⁰, —CO₂R⁶⁰, or —N(R⁶⁰)₂; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

49. The compound of claim 45, wherein:
R² and R^{2′} are independently alkyl; wherein said alkyl is C₁-C₆ alkyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and
each R⁶¹ independently is H, C₁-C₆ alkyl, phenyl, benzyl, morpholinyl, a 4-6 member β-lactam ring or cyclopentyl; wherein said 4-6 member β-lactam ring is substituted on a carbon or nitrogen atom with 2,4-dimethoxybenzyl; said cyclopentyl is optionally substituted with —OR⁶⁰ and said C₁-C₆ alkyl is optionally substituted with 1 to 3 moieties independently selected from the group consisting of phenyl, —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

50. The compound of claim 48, wherein said 5- to 6-membered heterocyclyl in R⁶¹ is morpholinyl, piperidinyl, pyrrolidinyl, or piperazinyl.

51. The compound of claim 45, wherein:
R² and R^{2′} are independently C₁-C₆ alkylsilyl;
R³ is —CN, —C(O)OR⁷ or —C(O)NR⁴R⁵; wherein said —C(O)OR⁷ is —C(O)OR⁶¹, and said —C(O)NR⁴R⁵ is —C(O)N(R⁶¹)₂; and each R⁶¹ independently is H, C₁-C₆ alkyl, phenyl, benzyl, morpholinyl, a 4-6 member β-lactam ring, or cyclopentyl, wherein said cyclopentyl is optionally substituted with —OR⁶⁰ and said C₁-C₆ alkyl is optionally substituted with —OR⁶⁰, —CO₂R⁶⁰, —CON(R⁶⁰)₂, —N(R⁶⁰)C(O)R⁶⁰, —N(R⁶⁰)C(O)-cyclopropyl, —N(R⁶⁰)₂, —N(R⁶⁰)C(O)OR⁶⁰, halo, —OC(O)N(R⁶⁰)₂, —CN, —N(R⁶⁰)C(O)N(R⁶⁰)₂, a 5- to 6-membered heterocyclyl optionally substituted with (═O), or —N(R⁶⁰)—CH₂-2-(6-tert-butyl-5,6,7,8-tetrahydro-thieno[2,3-b]quinolinyl); wherein said phenyl is optionally substituted with 1-2 moieties independently selected from the group consisting of —N(R⁶⁰)₂ and —N(R⁶⁰)C(O)R⁷⁰;
each R⁶⁰ independently is H or C₁-C₆ alkyl; and
R⁷⁰ is aryl or heteroaryl, wherein said aryl or heteroaryl is optionally substituted with 1-3 moieties independently selected from the group consisting of —CN, —OH, halo, C₁-C₆ alkyl, halo(C₁-C₆)alkyl, alkoxy, and —NR¹⁰R¹¹.

52. The compound of claim 1, selected from the group consisting of: 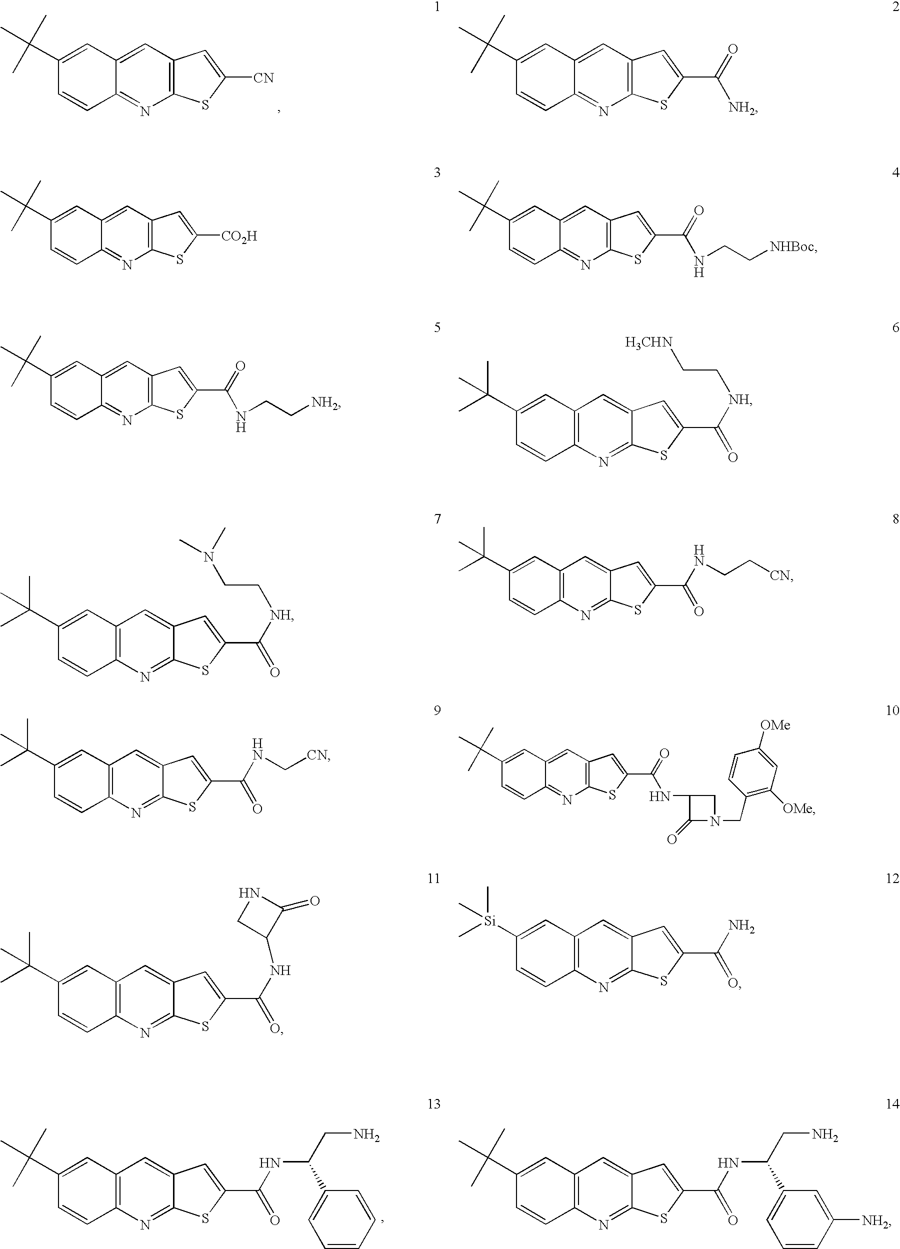 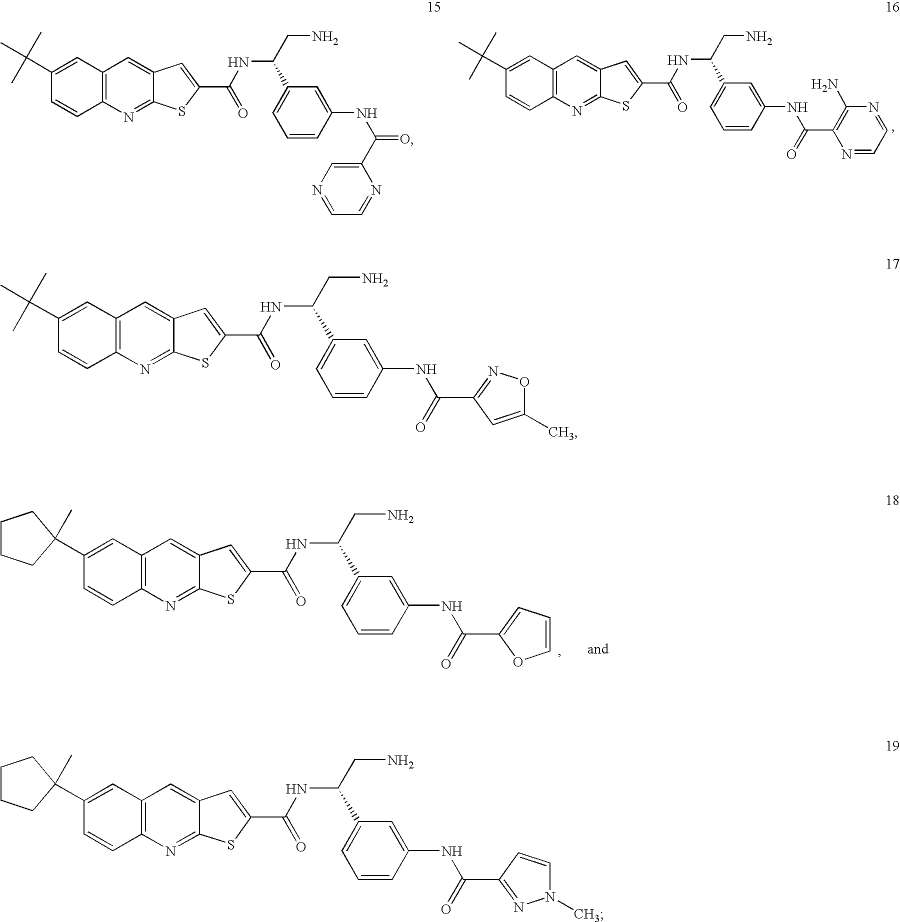
or a pharmaceutically acceptable salt or solvate thereof.

53. The compound of claim 52, wherein the compound is selected from the group consisting of: 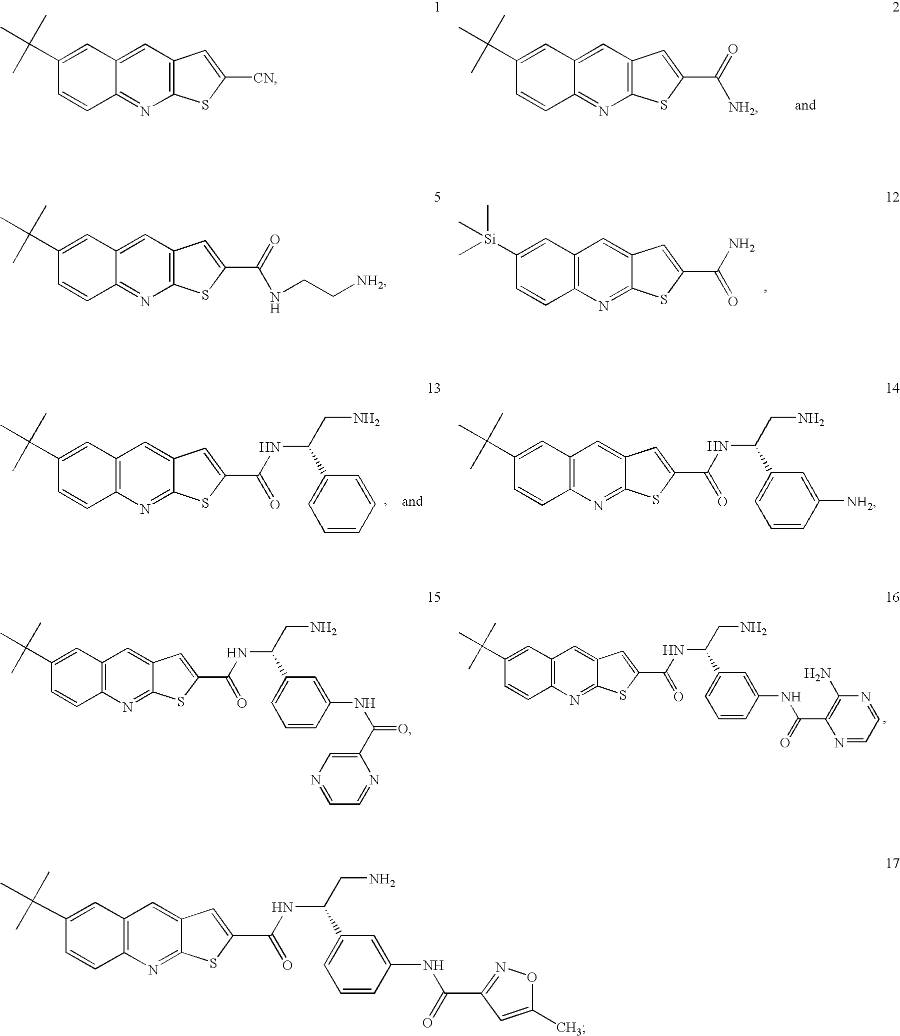
or a pharmaceutically acceptable salt or solvate thereof.

54. An isolated or purified form of a compound of claim 1.

55. A pharmaceutical composition comprising a therapeutically effective amount of at least one compound of claim 1, or a pharmaceutically acceptable salt or ester thereof, in combination with a pharmaceutically acceptable carrier.

56. The pharmaceutical composition of claim 55, further comprising one or more compounds selected from the group consisting of an anti-cancer agent, a PPAR-γ agonist, a PPAR-δ agonist, an inhibitor of inherent multidrug resistance, an anti-emetic agent, and an immunologic-enhancing drug.

57. The pharmaceutical composition of claim 56, wherein the anti-cancer agent is selected from the group consisting of an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, an inhibitor of cell proliferation and survival signaling, an agent that interfers with a cell cycle checkpoint, and an apoptosis inducing agent.

58. The pharmaceutical composition of claim 56, further comprising one or more anti-cancer agents selected from the group consisting of cytostatic agent, cytotoxic agent, taxane, topoisomerase II inhibitor, topoisomerase I inhibitor, tubulin interacting agent, hormonal agent, thymidilate synthase inhibitor, anti-metabolite, alkylating agent, farnesyl protein transferase inhibitor, signal transduction inhibitor, EGFR kinase inhibitor, antibodies to EGFR, C-abl kinase inhibitor, hormonal therapy combination, and aromatase combination.

59. The pharmaceutical composition of claim 56, further comprising one or more agents selected from the group consisting of Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin, cyclophosphamide, gemcitabine, interferons, pegylated interferons, Erbitux and mixtures thereof.

60. A method of inhibiting KSP activity in a subject in need thereof comprising administering to said subject an effective amount of at least one compound of claim 1 or a pharmaceutically acceptable salt or ester thereof.

61. A method of treating a cellular proliferative disease in a subject comprising administering to said subject in need of such treatment an effective amount of at least one compound claim 1 or a pharmaceutically acceptable salt or ester thereof.

62. The method of claim 61, wherein the cellular proliferative disease is selected from the group consisting of cancer, hyperplasia, cardiac hypertrophy, autoimmune diseases, fungal disorders, arthritis, graft rejection, inflammatory bowel disease, immune disorders, inflammation, and cellular proliferation induced after medical procedures.

63. The method of claim 62, wherein the cancer is selected from the group consisting of brain cancer, genitourinary tract cancer, cardiac cancer, gastrointestinal cancer, liver cancer, bone cancer, cancer of the nervous system, and lung cancer.

64. The method of claim 62, wherein the cancer is selected from lung adenocarcinama, small cell lung cancer, pancreatic cancer, and breast carcinoma.

65. The method of claim 62, further comprising administering radiation therapy to the subject.

66. The method of claim 62, further comprising administering to the subject at least one compound selected from the group consisting of an anti-cancer agent, a PPAR-γ agonist, a PPAR-δ agonist, an inhibitor of inherent multidrug resistance, an anti-emetic agent, and an immunologic-enhancing drug.

67. The method of claim 66, further comprising administering radiation therapy to the subject.

68. The method of claim 66, wherein the anti-cancer agent is selected from the group consisting of an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, an inhibitor of cell proliferation and survival signaling, an agent that interferes with a cell cycle checkpoint, and an apoptosis inducing agent.

69. The method of claim 62, further comprising administering to the subject one or more anti-cancer agents selected from the group consisting of cytostatic agent, cytotoxic agent, taxane, topoisomerase II inhibitor, topoisomerase I inhibitor, tubulin interacting agent, hormonal agent, thymidilate synthase inhibitor, anti-metabolite, alkylating agent, farnesyl protein transferase inhibitor, signal transduction inhibitor, EGFR kinase inhibitor, antibody to EGFR, C-abl kinase inhibitor, hormonal therapy combination, and aromatase combination.

70. The method of claim 62, further comprising administering to the subject one or more agents selected from the group consisting of Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin, cyclophosphamide, gemcitabine, interferons, pegylated interferons, Erbitux and mixtures thereof.
